(19) United States
(12) Patent Application Publication
(10) Pub. No.: US 2015/0044703 A1
(43) Pub. Date: Feb. 12, 2015

(54) METHODS AND COMPOSITIONS FOR DETECTING ENDOMETRIAL OR OVARIAN CANCER

(71) Applicant: UNIVERSITY OF SOUTH ALABAMA, Mobile, AL (US)

(72) Inventors: Michael A. Finan, Spanish Fort, AL (US); Rodney P. Rocconi, Mobile, AL (US); Lewis K. Pannel, Mobile, AL (US)

(73) Assignee: University of South Alabama, Mobile, AL (US)

(21) Appl. No.: 14/386,285

(22) PCT Filed: Mar. 12, 2013
(86) PCT No.: PCT/US2013/030590

§ 371 Date: Sep. 18, 2014

Related U.S. Application Data
(60) Provisional application No. 61/614,393, filed on Mar. 22, 2012.

(51) Int. Cl. G01N 33/574; H01J 49/00; C07K 14/47; C07K 16/18; C07K 7/08; C07K 7/06

(52) U.S. Cl. CPC G01N 33/57449; C07K 7/08; C07K 7/06; C07K 14/4748; C07K 16/18; G01N 33/57442; H01J 49/0031
USPC 435/7.92; 436/64; 436/518; 530/327; 530/328; 530/326; 530/324; 536/23.5; 530/387.9; 250/282

(57) ABSTRACT

Some embodiments of the present invention relate to methods and compositions for detecting the presence of cancer. In particular, methods and compositions for detecting endometrial cancer or ovarian cancer are provided.

### RELATED APPLICATIONS

This application claims the benefit of U.S. Provisional Application No. 61/614,393 entitled “METHODS AND COMPOSITIONS FOR DETECTING ENDOMETRIAL OR OVARIAN CANCER” filed Mar. 22, 2012, the contents of which is incorporated herein by reference in its entirety.

### REFERENCE TO SEQUENCE LISTING

The present application is being filed along with a Sequence Listing in electronic format. The Sequence Listing is provided as a file entitled SWFT_{—}006 WO.TXT, created Mar. 12, 2013, which is approximately 15 KB in size. The information in the electronic format of the Sequence Listing is incorporated herein by reference in its entirety.

### FIELD OF THE INVENTION

Some embodiments of the present invention relate to methods and compositions for detecting the absence, presence, progression, or stage of cancer. In particular, methods and compositions for detecting endometrial cancer or ovarian cancer are provided.

### BACKGROUND OF THE INVENTION

Ovarian cancer is among the most lethal gynecologic malignancies in developed countries. In the United States, approximately 23,000 women are diagnosed with the disease and almost 14,000 women die from it each year. There are three main types of ovarian cancer: epithelial, germ cell, and sex cord stromal. About 90% of ovarian cancers start in the epithelium tissue, which is the lining on the outside of the ovary. This type of ovarian cancer is divided into serous, mucinous, endometrioid, clear cell, transitional and undifferentiated types. The risk of epithelial ovarian cancer increases with age, especially after the age of 50. Germ cell tumors account for about 5% of ovarian cancers. They begin in the egg-producing cells. This type of ovarian cancer can occur in women of any age, but about 80% are found in women under the age of 30. The main subtypes are teratoma, dysgerminoma, endodermal sinus tumor and choriocarcinoma. Sex cord stromal tumors, about 5% of ovarian cancers, grow in the connective tissue that holds the ovary together and makes estrogen and progesterone. Most are found in older women. Despite progress in cancer therapy, ovarian cancer mortality has remained virtually unchanged over the past two decades. Given the steep survival gradient relative to the stage at which the disease is diagnosed, early detection remains the most important factor in improving long-term survival of ovarian cancer patients.

Endometrial cancer is the most common gynecologic malignancy and accounts for about 13% of all malignancies occurring in women. There are about 34,000 cases of endometrial cancer diagnosed in the United States each year. All endometrial carcinomas arise from the glands of the lining of the uterus. Adenocarcinoma accounts for 75% of all endometrial carcinoma. Endometrial adenocarcinomas that contain benign or malignant squamous cells are known as adenocanthomas and adenosquamous carcinomas respectively and account for 30% of endometrial cancers. The remaining types of endometrial carcinoma have a poorer prognosis. About 3% have a clear cell carcinoma, and about 1% have a papillary carcinoma.

Currently, there are no convincing early detection approaches for endometrial and ovarian cancers. Although it is well established that some endometrial and ovarian tumors shed cytologically recognizable cells in routinely prepared Pap tests, it is clear that this approach rarely detects occult tumors. Accordingly, efforts to develop means of collecting biological samples that have high patient acceptability, good sensitivity for detecting early disease, and excellent specificity are needed.

### SUMMARY OF THE INVENTION

Some embodiments of the methods and compositions provided herein include a method for determining the presence, absence, progression, or stage of a cancer in a female subject comprising determining the level of at least one polypeptide or fragment thereof or the level of at least one nucleic acid encoding said at least one polypeptide or a fragment thereof in a sample from said subject, wherein the polypeptide is selected from the group consisting of a polypeptide comprising, consisting essentially of, or consisting of SEQ ID NO.s:06-24, and 38-49.

In some embodiments, the sample is obtained from the cervix, the vagina, or the posterior vaginal fornix.

Some embodiments also include determining the level of at least one polypeptide comprising an amino acid sequence selected from the group consisting of a polypeptide comprising, consisting essentially of, or consisting of SEQ ID NO.s:01-05 and 25-37 or the level of at least one nucleic acids encoding said polypeptides or a fragment thereof.

Some embodiments also include determining the level of at least one polypeptide comprising an amino acid sequence selected from the group consisting of a polypeptide comprising, consisting essentially of, or consisting of SEQ ID NO.s:01-49 or the level of at least one nucleic acids encoding said polypeptides or a fragment thereof.

Some embodiments also include determining the level of at least two polypeptides comprising an amino acid sequence selected from the group consisting of a polypeptide comprising, consisting essentially of, or consisting of SEQ ID NO.s:01-49 or the level of at least two nucleic acids encoding said polypeptides or a fragment thereof.

Some embodiments also include determining the level of at least three polypeptides comprising an amino acid sequence selected from the group consisting of a polypeptide comprising, consisting essentially of, or consisting of SEQ ID NO.s:01-49 or the level of at least three nucleic acids encoding said polypeptides or a fragment thereof.

Some embodiments also include determining the level of at least five polypeptides comprising an amino acid sequence selected from the group consisting of a polypeptide comprising, consisting essentially of, or consisting of SEQ ID NO.s:01-49 or the level of at least five nucleic acids encoding said polypeptides or a fragment thereof.

Some embodiments also include comparing the level of at least one polypeptide or the level of a nucleic acid encoding the polypeptide in a sample from the subject with the level of at least polypeptide or the level of a nucleic acid encoding the polypeptide in a sample from a subject without the cancer.

In some embodiments, an increase in the level of said at least one polypeptide or fragment thereof or the level of said at least one nucleic acid encoding the polypeptide or a fragment thereof in a sample from said subject compared to the level of said at least one polypeptide or fragment thereof or the level of said at least one nucleic acid encoding said at least one polypeptide in a sample from said subject without cancer is indicative of the presence of the cancer in the subject.

In some embodiments, at least one polypeptide is selected from the group consisting of a polypeptide comprising, consisting essentially of, or consisting of SEQ ID NO.s: 1-5, 7-19, 21, 23-45, and 47-48.

In some embodiments, the cancer comprises endometrial cancer, wherein the polypeptide is selected from the group consisting of a polypeptide comprising, consisting essentially of, or consisting of SEQ ID NO.s: 1-5, 7-19, 21, 23-24.

In some embodiments, the cancer comprises ovarian cancer, wherein the polypeptide is selected from the group consisting of a polypeptide comprising, consisting essentially of, or consisting of SEQ ID NO.s: 25-45, and 47-48.

In some embodiments, at least a 3-fold increase in the level of the said at least one polypeptide or fragment thereof or the level of said at least one nucleic acid encoding the polypeptide or fragment thereof in a sample from said subject compared to the level of said at least one polypeptide or fragment thereof or the level of said at least one nucleic acid encoding the polypeptide or fragment thereof in a sample from said subject without cancer is indicative of the presence of the cancer in the subject.

In some embodiments, at least a 5-fold increase in the level of the said at least one polypeptide or fragment thereof or the level of said at least one nucleic acid encoding the polypeptide or fragment thereof in a sample from said subject compared to the level of said at least one polypeptide or fragment thereof or the level of said at least one nucleic acid encoding the polypeptide or fragment thereof in a sample from said subject without cancer is indicative of the presence of the cancer in the subject.

In some embodiments, at least a 10-fold increase in the level of the said at least one polypeptide or fragment thereof or the level of said at least one nucleic acid encoding the polypeptide or fragment thereof in a sample from said subject compared to the level of said at least one polypeptide or fragment thereof or the level of said at least one nucleic acid encoding the polypeptide or fragment thereof in a sample from said subject without cancer is indicative of the presence of the cancer in the subject.

In some embodiments, at least a 100-fold increase in the level of the said at least one polypeptide or fragment thereof or the level of said at least one nucleic acid encoding the polypeptide or fragment thereof in a sample from said subject compared to the level of said at least one polypeptide or fragment thereof or the level of said at least one nucleic acid encoding the polypeptide or fragment thereof in a sample from said subject without cancer is indicative of the presence of the cancer in the subject.

In some embodiments, a decrease in the level of said at least one polypeptide or fragment thereof or the level of said at least one nucleic acid encoding the polypeptide or a fragment thereof in a sample from said subject compared to the level of said at least one polypeptide or fragment thereof or the level of said at least one nucleic acid encoding said at least one polypeptide in a sample from said subject without cancer is indicative of the presence of the cancer in the subject.

In some embodiments, the at least one polypeptide is selected from the group consisting of a polypeptide comprising, consisting essentially of, or consisting of SEQ ID NO.s: 6, 20, 22, and 46

In some embodiments, the cancer comprises endometrial cancer, wherein the at least one polypeptide is selected from the group consisting of a polypeptide comprising, consisting essentially of, or consisting of SEQ ID NO.s: 6, 20, and 22.

In some embodiments, the cancer comprises ovarian cancer, wherein the at least one polypeptide is SEQ ID NO.: 46

In some embodiments, at least a 3-fold decrease in the level of the said at least one polypeptide or fragment thereof or the level of said at least one nucleic acid encoding the polypeptide or fragment thereof in a sample from said subject compared to the level of said at least one polypeptide or fragment thereof or the level of said at least one nucleic acid encoding the polypeptide or fragment thereof in a sample from said subject without cancer is indicative of the presence of the cancer in the subject.

In some embodiments, at least a 5-fold decrease in the level of the said at least one polypeptide or fragment thereof or the level of said at least one nucleic acid encoding the polypeptide or fragment thereof in a sample from said subject compared to the level of said at least one polypeptide or fragment thereof or the level of said at least one nucleic acid encoding the polypeptide or fragment thereof in a sample from said subject without cancer is indicative of the presence of the cancer in the subject.

In some embodiments, at least a 10-fold decrease in the level of the said at least one polypeptide or fragment thereof or the level of said at least one nucleic acid encoding the polypeptide or fragment thereof in a sample from said subject compared to the level of said at least one polypeptide or fragment thereof or the level of said at least one nucleic acid encoding the polypeptide or fragment thereof in a sample from said subject without cancer is indicative of the presence of the cancer in the subject.

In some embodiments, determining the level of said at least one polypeptide or fragment thereof comprises performing an immunoassay or a colorimetric assay.

In some embodiments, the immunoassay is selected from the group consisting of a Western blot, an enzyme linked immunoabsorbent assay (ELISA), and radioimmunoassay.

In some embodiments, determining the level of said at least one polypeptide or fragment thereof comprises mass spectrometry.

In some embodiments, determining the level of said at least one polypeptide or fragment thereof comprises applying said sample to a solid phase test strip or a flow-through strip comprising an agent which selectively binds to said at least one polypeptide or fragment thereof; and detecting said polypeptide bound to said agent on said solid phase test strip or said flow-through strip.

In some embodiments, the cancer is a non-cervical cancer of the gynecological tract.

In some embodiments, the cancer is selected from the group consisting of endometrial cancer, and ovarian cancer.

In some embodiments, the cancer is selected from the group consisting of endometrial hyperplasia, endometrial hyperplasia with atypia, and non-invasive endometrial cancer.

In some embodiments, the sample is obtained from a cervical pap specimen.

In some embodiments, the sample is substantially free of cells.

In some embodiments, the at least one polypeptide comprises a protein selected from the group consisting of mesotrypsin isoform 2, apolipoprotein A-I, transferring, alpha-1b-glycoprotein, hemopexin, alpha 2 globin, serine proteinase inhibitor: clade A: member 1, keratin 6C, profilin 1, periplakin, and calcium-binding protein A8 or fragment thereof.

In some embodiments, the subject is human.

Some embodiments of the methods and compositions provided herein include a kit for determining the presence, absence, progression, or stage of a cancer in a female subject comprising (a) a suitable diluent for irrigating the uterine cavity of the subject; (b) a receptacle for collection of the diluted uterine fluid; and (c) an agent that selectively binds to at least one polypeptide or nucleic acid encoding a polypeptide, wherein said polypeptide comprises an amino acid sequence selected from the group consisting of a polypeptide comprising, consisting essentially of, or consisting of SEQ ID NO.s:06-24, and 38-49.

Some embodiments also include an agent that selectively binds to at least one polypeptide or nucleic acid encoding a polypeptide, wherein said polypeptide is selected from the group consisting of a polypeptide comprising, consisting essentially of, or consisting of SEQ ID NO.s:01-05 and 25-37.

Some embodiments also include an agent that selectively binds to at least one polypeptide or nucleic acid encoding a polypeptide, wherein said polypeptide is selected from the group consisting of a polypeptide comprising, consisting essentially of, or consisting of SEQ ID NO.s:01-49.

Some embodiments also include at least three agents that each selectively bind to a different polypeptide or a nucleic acid encoding said polypeptide.

Some embodiments also include at least five agents that each selectively bind to a different polypeptide or a nucleic acid encoding said polypeptide

In some embodiments, the agent comprises an antibody or antigen-binding fragment thereof.

In some embodiments, the at least one polypeptide comprises a protein selected from the group consisting of mesotrypsin isoform 2, apolipoprotein A-I, transferring, alpha-1b-glycoprotein, hemopexin, alpha 2 globin, serine proteinase inhibitor: clade A: member 1, keratin 6C, profilin 1, periplakin, and calcium-binding protein A8 or fragment thereof.

Some embodiments of the methods and compositions provided herein include a kit comprising an agent which selectively binds to at least one polypeptide comprising an amino acid sequence selected from the group consisting of a polypeptide comprising, consisting essentially of, or consisting of SEQ ID NO.s:06-24, and 38-49 or a fragment thereof, wherein said agent is attached to a solid support.

Some embodiments also include an agent that selectively binds to at least one polypeptide or nucleic acid encoding a polypeptide, wherein said polypeptide is selected from the group consisting of a polypeptide comprising, consisting essentially of, or consisting of SEQ ID NO.s:01-05 and 25-37.

Some embodiments also include an agent that selectively binds to at least one polypeptide or nucleic acid encoding a polypeptide, wherein said polypeptide is selected from the group consisting of a polypeptide comprising, consisting essentially of, or consisting of SEQ ID NO.s:01-49.

In some embodiments, a plurality of agents that bind to different polypeptides comprising an amino acid sequence selected from the group consisting of a polypeptide comprising, consisting essentially of, or consisting of SEQ ID NO.s:01-49 or a fragment thereof are attached to said solid support.

In some embodiments, the solid support comprises a solid phase test strip or a flow-through test strip.

Some embodiments also include a detectable agent which selectively binds to said polypeptide.

In some embodiments, the at least one polypeptide comprises a protein selected from the group consisting of mesotrypsin isoform 2, apolipoprotein A-I, transferring, alpha-1b-glycoprotein, hemopexin, alpha 2 globin, serine proteinase inhibitor: clade A: member 1, keratin 6C, profilin 1, periplakin, and calcium-binding protein A8 or fragment thereof.

Some embodiments of the methods and compositions provided herein include a kit comprising an agent which selectively binds to at least one nucleic acid encoding a polypeptide comprising an amino acid sequence selected from the group consisting of a polypeptide comprising, consisting essentially of, or consisting of SEQ ID NO.s:06-24, and 38-49 or a fragment thereof, wherein said agent is attached to a solid support.

Some embodiments also include an agent that selectively binds to at least one polypeptide or nucleic acid encoding a polypeptide, wherein said polypeptide is selected from the group consisting of a polypeptide comprising, consisting essentially of, or consisting of SEQ ID NO.s:01-05 and 25-37.

Some embodiments also include an agent that selectively binds to at least one polypeptide or nucleic acid encoding a polypeptide, wherein said polypeptide is selected from the group consisting of a polypeptide comprising, consisting essentially of, or consisting of SEQ ID NO.s:01-49.

In some embodiments, a plurality of agents that bind to nucleic acids encoding different polypeptides comprising an amino acid sequence selected from the group consisting of a polypeptide comprising, consisting essentially of, or consisting of SEQ ID NO.s:01-49 or a fragment thereof are attached to said solid support.

In some embodiments, the solid support comprises a solid phase test strip or a flow-through test strip.

Some embodiments also include a detectable agent which selectively binds to said polypeptide.

In some embodiments, the at least one polypeptide comprises a protein selected from the group consisting of mesotrypsin isoform 2, apolipoprotein A-I, transferring, alpha-1b-glycoprotein, hemopexin, alpha 2 globin, serine proteinase inhibitor: clade A: member 1, keratin 6C, profilin 1, periplakin, and calcium-binding protein A8 or fragment thereof.

In some embodiments, the cancer is selected from the group consisting of endometrial cancer, and ovarian cancer.

In some embodiments, the cancer is selected from the group consisting of endometrial hyperplasia, endometrial hyperplasia with atypia, and non-invasive endometrial cancer.

Some embodiments of the methods and compositions provided herein include an isolated polypeptide consisting essentially of an amino acid sequence selected from the group consisting of a polypeptide comprising, consisting essentially of, or consisting of SEQ ID NO.s:06-24, and 38-49 or a fragment thereof, wherein said polypeptide is differentially expressed in cancer.

Some embodiments of the methods and compositions provided herein include an isolated nucleic acid encoding a polypeptide consisting essentially of an amino acid sequence selected from the group consisting of a polypeptide comprising, consisting essentially of, or consisting of SEQ ID NO.s:06-24, and 38-49 or a fragment thereof, wherein said polypeptide is differentially expressed in cancer.

Some embodiments of the methods and compositions provided herein include an isolated polypeptide consisting of an amino acid sequence selected from the group consisting of a polypeptide comprising, consisting essentially of, or consisting of SEQ ID NO.s:06-24, and 38-49 or a fragment thereof, wherein said polypeptide is differentially expressed in cancer.

Some embodiments of the methods and compositions provided herein include an isolated nucleic acid encoding a polypeptide consisting of an amino acid sequence selected from the group consisting of a polypeptide comprising, consisting essentially of, or consisting of SEQ ID NO.s:06-24, and 38-49 or a fragment thereof, wherein said polypeptide is differentially expressed in cancer.

Some embodiments of the methods and compositions provided herein include an isolated agent that selectively binds to an isolated polypeptide consisting essentially of an amino acid sequence selected from the group consisting of a polypeptide comprising, consisting essentially of, or consisting of SEQ ID NO.s:06-24, and 38-49 or a fragment thereof, wherein said polypeptide is differentially expressed in cancer.

In some embodiments, the agent comprises an antibody or antigen-binding fragment thereof.

Some embodiments of the methods and compositions provided herein include an isolated agent that selectively binds to an isolated polypeptide consisting of an amino acid sequence selected from the group consisting of a polypeptide comprising, consisting essentially of, or consisting of SEQ ID NO.s:06-24, and 38-49 or a fragment thereof, wherein said polypeptide is differentially expressed in cancer.

In some embodiments, the agent comprises an antibody or antigen-binding fragment thereof.

### BRIEF DESCRIPTION OF THE DRAWINGS

FIG. 1 shows a cluster for 32 peptides selected from peptides with least 100 FDR Wilcoxon test FDR p-value 0.05 and AUC greater than 0.80.

FIG. 2 shows a cluster for 50 peptides selected from peptides with Wilcoxon test FDR p-value 0.05.

FIG. 3 shows clustering of albumin peptides. Clustering was based on similarity (r2) between albumin peptides. Those peptides that exhibited similar information clustered together. Peptides at masses 1169 (SEQ ID NO:35), 1303 (SEQ ID NO:36), and 1757 (SEQ ID NO:32), provide nearly identical information for any regression analysis.

FIG. 4 show graphs of m/z vs. intensity (top panel) or observed calculated m/z (bottom panel), with regards to SEQ ID NO:01 iron-modified at residue E4.

FIG. 5 show graphs of m/z vs. intensity (top panel) or observed calculated m/z (bottom panel), with regards to SEQ ID NO:01 iron-modified at residue E10.

FIG. 6 show graphs of m/z vs. intensity (top panel) or observed calculated m/z (bottom panel), with regards to SEQ ID NO:01 iron-modified at residue E11.

FIG. 7 depicts an example MASCOT search result.

### DETAILED DESCRIPTION

Some embodiments of the present invention relate to methods and compositions for detecting the presence of cancer. In particular, methods and compositions for detecting endometrial cancer or ovarian cancer are provided.

Applicant has discovered that certain target molecules in samples are useful in the diagnosis of cancer. In particular embodiments, the cancer is endometrial cancer or ovarian cancer. Examples of the target molecules include certain polypeptides and fragments thereof, and nucleic acids encoding such polypeptides and fragments thereof. In some embodiments, the samples originate from the cervix, the vagina, or the posterior vaginal fornix of a subject. In some embodiments, the samples are obtained using methods described in U.S. application Ser. No. 12/646,592, entitled “NOVEL MOLECULAR ASSAY AND USES THEREOF”, the disclosure of which is incorporated herein by reference in its entirety. Examples include mesotrypsin isoform 2, apolipoprotein A-I, transferring, alpha-1b-glycoprotein, hemopexin, alpha 2 globin, serine proteinase inhibitor: clade A: member 1, keratin 6C, profilin 1, periplakin, and calcium-binding protein A8.

Proteomic analysis of body fluids can yield information for biomarker discovery and treatment development. In some embodiments, the body fluids are cervico-vaginal fluids. Cervico-vaginal fluid samples are especially interesting in terms of gynecological diagnostics since these samples can easily be collected using non-invasive methods. Although conventional biomarkers are often quantified in plasma samples, there are two reasons why cervico-vaginal fluid samples are preferred over plasma samples in terms of gynecological biomarker discovery. First, since the volume of plasma (about 3 liters) is much larger than e.g. vaginal washings (about 50 ml), it could be expected that dilution of a (potential) biomarker will be much lower in the latter fluid. Second, altered biomarker expression patterns in plasma are often not very specific as they may be associated with different pathologies because plasma comes in contact with all organs of the body. In contrast, when using cervico-vaginal fluid samples, it is expected that expression patterns will directly correlate with gynecological pathologies.

### Biological Sample

A biological sample can include any body fluid or tissue. Preferred body fluids include blood, plasma, serum, urine, saliva, sputum, cerebrospinal fluid, mucus, and vaginal and rectal secretions; preferably the biological sample includes blood or blood products such as plasma and serum. Embodiments provided herein are directed toward the analysis of cancer, in particular, endometrial and ovarian cancers, tissues and fluids originating from the uterus, cervix, vagina and the like are preferred. When tissue samples are used, such as biopsies, they can be homogenized, for example in phosphate buffered saline or, alternatively, in a detergent-containing buffer to solubilize the polypeptides to be detected.

### Sample Processing

In some embodiments, a test sample can be preprocessed prior to analysis of its protein content, for example to remove nonproteinaceous sample components. Methods for preprocessing include, without limitation, various forms of chromatography (size exclusion, hydrophobic, ion exchange, affinity and the like), microfiltration, centrifugation and dialysis. Preprocessing also can include subjecting the sample to chemical or enzymatic protein cleavage agents in order to break down the proteins into smaller components. Additionally or alternatively, the test sample is optionally fractionated into subsamples, each containing a subset of sample proteins, prior to analyzing the sample for polypeptide biomarkers.

The amount of a target molecule, such as a polypeptide or fragment thereof, in the test sample or a control sample can be zero, in which case “amount” refers to the presence or absence of the target molecule, which presence or absence is indicative of a cancer. Alternatively, the target molecule can be present in both samples, but at a higher (upregulated) or lower (downregulated) level in the test sample which is indicative of cancer.

Amounts of target molecules can be determined in absolute or relative terms. If expressed in relative terms, amounts can be expressed as normalized amounts with reference to a selected target molecule present in the sample.

In some embodiments, after optional preprocessing and/or fractionation, target molecules are physically separated prior to determining the amounts of each target molecules. Physical separation can be achieved, for example, using single or multidimensional chromatography, electrochromatography or electrophoresis, such as 2D electrophoresis. The amount of the separated target molecules can be determined using any convenient method such as spectroscopic (e.g., UV detection) or colorimetric (e.g., staining) methods. Optionally, the identity of separated target molecules of interest can be determined using standard techniques such as protein sequencing and tandem mass spectrometry.

In other embodiments of the invention, after optional preprocessing and/or fractionation, sample components are not further separated but instead the sample is subjected to mass analysis, for example using peptide-mass fingerprinting or mass spectrometry.

### Methods for Detecting Target Molecules

Target molecules can be detected by any means known in the art. By way of non-limiting example, polypeptide target molecules may be detected by using immunohistological, immunocytological, hybridization using immunofluorescence and/or immunoenzymatic, techniques as well as hydrometry, polarimetry, spectrophotometry (e.g., mass and NMR) and chromatography (e.g., gas liquid, high performance liquid, and thin layer). In some embodiments, nucleic acid target molecules may be detected using nucleic acid hybridization methods, such as Southern blotting, Northern blotting, or PCR.

Some embodiments of the methods and compositions provided herein include characterizing a target molecule in a sample, such as a sample obtained from the cervix, the vagina, or the posterior vaginal fornix. Characterizing a target molecule can include, for example, identifying a target molecule, detecting a target molecule, and/or quantifying a target molecule. Methods to identify, detect and quantify target molecule are well known in the art.

Some embodiments include identifying, determining the presence or absence of a target molecule, and/or quantifying a target molecule, wherein the target molecule comprises a peptide, polypeptide, and/or protein.

As used in the present specification, the term “polypeptide” and “protein”, used interchangeably herein, refer to a polymer of amino acids without regard to the length of the polymer; thus, peptides, oligopeptides, and proteins are included within the definition of polypeptide. This term also includes wild-type polypeptides, as well as mutants, truncations, extensions, splice-variants, and other non-native forms of polypeptide that may be present. This term also includes forms of the foregoing that have been subject to enzymatic degradation by proteases or other mechanisms (enzymatic or non-enzymatic) in the subject. For example, a polypeptide may be subject to degradation by a protease to produce a polypeptide fragment of the polypeptide. The protease may be one that is expressed or increased in expression as a result of the health problem or disease of the gynecological system. The polypeptide may have been originally on a cellular surface but proteolytically processed or removed as a result of a disease process and collected into the mucus. This term also does not specify or exclude chemical or post-expression/translational modifications of the polypeptides, although chemical or post-expression modifications of these polypeptides may be included or excluded as specific embodiments. Therefore, for example, modifications to polypeptides that include the covalent attachment of glycosyl groups (i.e., glycosylation), acetyl groups (i.e., acetylation), phosphate groups (phosphorylation, including, but not limited to, phosphorylation on serine, threonine and tyrosine groups), lipid groups and the like are expressly encompassed by the term polypeptide. Further, polypeptides with these modifications may be specified as individual species to be included or excluded. The natural or other chemical modifications, such as those listed in examples above can occur anywhere in a polypeptide, including the peptide backbone, the amino acid side-chains and the amino or carboxyl termini, and may be present in the same or varying degrees at several sites in a given polypeptide. Also, a given polypeptide may contain many types of modifications. Polypeptides may be branched, for example, as a result of ubiquitination, and they may be cyclic, with or without branching. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formylation of cysteine, formylation of pyroglutamate, formylation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination (see, for instance Creighton, (1993), Posttranslational Covalent Modification of Proteins, W. H. Freeman and Company, New York B. C. Johnson, Ed., Academic Press, New York 1-12; Seifier, et aI., (1990) Meth EnzymoI182:626-646; Rattan et aI, (1992) Ann NY Acad Sci 663:48-62).

Such target polypeptide molecules may be characterized by a variety of methods such as immunoassays, including radioimmunoassays, enzyme-linked immunoassays and two-antibody sandwich assays as described herein. A variety of immunoassay formats, including competitive and non-competitive immunoassay formats, antigen capture assays and two-antibody sandwich assays also are useful (Self and Cook, (1996) Curr. Opin. Biotechnol. 7:60-65, incorporated by reference in its entirety). Some embodiments include one or more antigen capture assays. In an antigen capture assay, antibody is bound to a solid phase, and sample is added such that antigen, e.g., a target molecule in a fluid or tissue sample, is bound by the antibody. After unbound proteins are removed by washing, the amount of bound antigen can be quantitated, if desired, using, for example, a radioassay (Harlow and Lane, (1988) Antibodies A Laboratory Manual Cold Spring Harbor Laboratory: New York, incorporated by reference in its entirety). Immunoassays can be performed under conditions of antibody excess, or as antigen competitions, to quantitate the amount of antigen and, thus, determine a level of a target molecule in a sample, such as a sample obtained from the cervix, the vagina, or the posterior vaginal fornix.

Enzyme-linked immunosorbent assays (ELISAs) can be useful in certain embodiments provided herein. An enzyme such as horseradish peroxidase (HRP), alkaline phosphatase (AP), β-galactosidase or urease can be linked, for example, to an anti-HMGB1 antibody or to a secondary antibody for use in a method of the invention. A horseradish-peroxidase detection system can be used, for example, with the chromogenic substrate tetramethylbenzidine (TMB), which yields a soluble product in the presence of hydrogen peroxide that is detectable at 450 nm. Other convenient enzyme-linked systems include, for example, the alkaline phosphatase detection system, which can be used with the chromogenic substrate p-nitrophenyl phosphate to yield a soluble product readily detectable at 405 nm. Similarly, a β-galactosidase detection system can be used with the chromogenic substrate o-nitrophenyl-β-D-galactopyranoside (ONPG) to yield a soluble product detectable at 410 nm, or a urease detection system can be used with a substrate such as urea-bromocresol purple (Sigma Immunochemicals). Useful enzyme-linked primary and secondary antibodies can be obtained from a number of commercial sources such as Jackson Immuno-Research (West Grove, Pa.) as described further herein.

In certain embodiments, a target molecule in a sample, such as a sample obtained from the cervix, the vagina, or the posterior vaginal fornix, can be detected and/or measured using chemiluminescent detection. For example in certain embodiments, specific antibodies to a particular target molecule are used to capture the target molecule present in the biological sample, e.g., such as a sample obtained from the cervix, the vagina, or the posterior vaginal fornix, and an antibody specific for the target molecule-specific antibodies and labeled with an chemiluminescent label is used to detect the target molecule present in the sample. Any chemiluminescent label and detection system can be used in the present methods. Chemiluminescent secondary antibodies can be obtained commercially from various sources such as Amersham. Methods of detecting chemiluminescent secondary antibodies are known in the art.

Fluorescent detection also can be useful for detecting a target molecule in certain methods provided herein. Useful fluorochromes include, DAPI, fluorescein, Hoechst 33258, R-phycocyanin, B-phycoerythrin, R-phycoerythrin, rhodamine, Texas red and lissamine. Fluorescein or rhodamine labeled antibodies, or fluorescein- or rhodamine-labeled secondary antibodies can be useful in the invention.

Radioimmunoassays (RIAs) also can be useful in certain methods provided herein. Such assays are well known in the art. Radioimmunoassays can be performed, for example, with ¹²⁵I-labeled primary or secondary antibody (Harlow and Lane, (1988) Antibodies A Laboratory Manual Cold Spring Harbor Laboratory: New York, incorporated by reference in its entirety).

A signal from a detectable reagent can be analyzed, for example, using a spectrophotometer to detect color from a chromogenic substrate; a radiation counter to detect radiation, such as a gamma counter for detection of ¹²⁵I; or a fluorometer to detect fluorescence in the presence of light of a certain wavelength. Where an enzyme-linked assay is used, quantitative analysis of the amount of a target molecule can be performed using a spectrophotometer such as an EMAX Microplate Reader (Molecular Devices; Menlo Park, Calif.) in accordance with the manufacturer's instructions. The assays of the invention can be automated or performed robotically, if desired, and that the signal from multiple samples can be detected simultaneously.

In some embodiments, capillary electrophoresis based immunoassays (CEIA), which can be automated if desired, may be used to detect and/or measure the target molecule. Immunoassays also can be used in conjunction with laser-induced fluorescence as described, for example, in Schmalzing and Nashabeh, Electrophoresis 18:2184-93 (1997), and Bao, J. Chromatogr. B. Biomed. Sci. 699:463-80 (1997), each incorporated by reference in its entirety. Liposome immunoassays, such as flow-injection liposome immunoassays and liposome immunosensors, also can be used to detect target molecules or to determine a level of a target molecule according to certain methods provided herein (Rongen et al., (1997) J. Immunol. Methods 204:105-133, incorporated by reference in its entirety).

Sandwich enzyme immunoassays also can be useful in certain embodiments. In a two-antibody sandwich assay, a first antibody is bound to a solid support, and the antigen is allowed to bind to the first antibody. The amount of a target molecule is quantitated by measuring the amount of a second antibody that binds to it.

In an example sandwich assay, an agent that selectively binds to a target molecule can be immobilized on a solid support. A capture reagent can be chosen to directly bind the target molecule or indirectly bind the target molecule by binding with an ancillary specific binding member which is bound to the target molecule. In addition, the capture reagent may be immobilized on the solid phase before or during the performance of the assay by means of any suitable attachment method. Typically, the capture site of the present invention is a delimited or defined portion of the solid phase such that the specific binding reaction of the capture reagent and analyte is localized or concentrated in a limited site, thereby facilitating the detection of label that is immobilized at the capture site in contrast to other portions of the solid phase. In a related embodiment, the capture reagent can be applied to the solid phase by dipping, inscribing with a pen, dispensing through a capillary tube, or through the use of reagent jet-printing or other techniques. In addition, the capture zone can be marked, for example with a dye, such that the position of the capture zone upon the solid phase can be visually or instrumentally determined even when there is no label immobilized at the site.

Another example embodiment of a sandwich assay format includes methods and compositions wherein a sample is mixed with a labeled first specific binding pair member for the target molecule and allowed to traverse a lateral flow matrix, past a series of spatially separated capture zones located on the matrix (See e.g., U.S. Pat. No. 7,491,551, incorporated by reference in its entirety). The sample may be mixed with the labeled first specific binding pair member prior to addition of the sample to the matrix. Alternatively, the labeled first specific binding pair member may be diffusively bound on the matrix on a labeling zone at a point upstream of the series of capture zones. Sometimes, the sample is added directly to the labeling zone. Preferably, the sample is added to a sample receiving zone on the matrix at a point upstream of the labeling zone and allowed to flow through the labeling zone. The labeled first specific binding pair member located within the labeling zone is capable of being freely suspendable in the sample. Therefore, if analyte is present in the sample, the labeled first specific binding pair member will bind to the target molecule and the resulting target molecule-labeled first specific binding pair member complex will be transported to and through the capture zones. The extent of complex formation between the target molecule and the labeled specific binding pair member is, directly proportional to the amount of target molecule present in the sample. A second specific binding pair member capable of binding to the target molecule-first specific binding pair member complex is immobilized on each of the capture zones. This second specific binding pair member is not capable of binding the labeled specific binding pair member unless the labeled specific binding pair member is bound to the target molecule. Thus, the amount of labeled specific binding pair member that accumulates on the capture zones is directly proportional to the amount of target molecule present in the sample.

In some embodiments, an assay includes the use of binding agent immobilized on a solid support to bind to and remove a target polypeptide from the remainder of the sample. The bound target polypeptide may then be detected using a detection reagent that contains a reporter group and specifically binds to the binding agent/polypeptide complex. Such detection reagents may comprise, for example, a binding agent that specifically binds to the target polypeptide or an antibody or other agent that specifically binds to the binding agent, such as an anti-immunoglobulin, protein G, protein A or a lectin. In such embodiments, the binding agent can comprise an antibody or antigen-binding fragment thereof specific to a polypeptide or fragment thereof descried herein. Alternatively, a competitive assay may be utilized, in which a polypeptide is labeled with a reporter group and allowed to bind to the immobilized binding agent after incubation of the binding agent with the sample. The extent to which components of the sample inhibit the binding of the labeled polypeptide to the binding agent is indicative of the reactivity of the sample with the immobilized binding agent. Suitable polypeptides for use within such assays include full length proteins provided herein and polypeptide portions thereof such as SEQ ID NO: 1-49 to which the binding agent binds.

The solid support may be any material known to those of ordinary skill in the art to which the binding agent may be attached. For example, the solid support may be a test well in a microtiter plate or a nitrocellulose or other suitable membrane or flow-through format or test strip. Alternatively, the support may be a bead or disc, such as glass, fiberglass, latex or a plastic material such as polystyrene or polyvinylchloride. The support may also be a magnetic particle or a fiber optic sensor, such as those disclosed, for example, in U.S. Pat. No. 5,359,681. The binding agent may be immobilized on the solid support using a variety of techniques known to those of skill in the art, which are amply described in the patent and scientific literature. In the context of the present invention, the term “immobilization” refers to both noncovalent association, such as adsorption, and covalent attachment (which may be a direct linkage between the agent and functional groups on the support or may be a linkage by way of a cross-linking agent). Immobilization by adsorption to a well in a microtiter plate or to a membrane is preferred. In such cases, adsorption may be achieved by contacting the binding agent, in a suitable buffer, with the solid support for a suitable amount of time. The contact time varies with temperature, but is typically between about 1 hour and about 1 day. In general, contacting a well of a plastic microtiter plate (such as polystyrene or polyvinylchloride) with an amount of binding agent ranging from about 10 ng to about 10 μg, and preferably about 100 ng to about 1 μg, is sufficient to immobilize an adequate amount of binding agent.

Covalent attachment of binding agent to a solid support may generally be achieved by first reacting the support with a bifunctional reagent that will react with both the support and a functional group, such as a hydroxyl or amino group, on the binding agent. For example, the binding agent may be covalently attached to supports having an appropriate polymer coating using benzoquinone or by condensation of an aldehyde group on the support with an amine and an active hydrogen on the binding partner (see, e.g., Pierce Immunotechnology Catalog and Handbook, 1991, at A12-A13).

In certain embodiments, the assay is a two-antibody sandwich assay. This assay may be performed by first contacting an antibody that has been immobilized on a solid support, commonly the well of a microtiter plate, with the sample, such that target polypeptides within the sample are allowed to bind to the immobilized antibody. Unbound sample is then removed from the immobilized polypeptide-antibody complexes and a detection reagent (preferably a second antibody capable of binding to a different site on the polypeptide) containing a reporter group is added. The amount of detection reagent that remains bound to the solid support is then determined using a method appropriate for the specific reporter group.

More specifically, once the antibody is immobilized on the support as described above, the remaining protein binding sites on the support are typically blocked. Any suitable blocking agent known to those of ordinary skill in the art may be used, such as bovine serum albumin or TWEEN 20. (Sigma Chemical Co., St. Louis, Mo.). The immobilized antibody is then incubated with the sample, and target polypeptide is allowed to bind to the antibody. The sample may be diluted with a suitable diluent, such as phosphate-buffered saline (PBS) prior to incubation. In general, an appropriate contact time (i.e., incubation time) is a period of time that is sufficient to detect the presence of target polypeptide within a sample obtained from an individual with breast cancer. Preferably, the contact time is sufficient to achieve a level of binding that is at least about 95% of that achieved at equilibrium between bound and unbound polypeptide. Those of ordinary skill in the art will recognize that the time necessary to achieve equilibrium may be readily determined by assaying the level of binding that occurs over a period of time. At room temperature, an incubation time of about 30 minutes is generally sufficient.

Unbound sample may then be removed by washing the solid support with an appropriate buffer, such as PBS containing 0.1% TWEEN 20. The second antibody, which contains a reporter group, may then be added to the solid support. Reporter groups are well known in the art. The detection reagent is then incubated with the immobilized antibody-polypeptide complex for an amount of time sufficient to detect the bound detection reagent. An appropriate amount of time may generally be determined by assaying the level of binding that occurs over a period of time. Unbound detection reagent is then removed and bound detection reagent is detected using the reporter group. The method employed for detecting the reporter group depends upon the nature of the reporter group. For radioactive groups, scintillation counting or autoradiographic methods are generally appropriate. Spectroscopic methods may be used to detect dyes, luminescent groups and fluorescent groups. Biotin may be detected using avidin, coupled to a different reporter group (commonly a radioactive or fluorescent group or an enzyme). Enzyme reporter groups may generally be detected by the addition of substrate (generally for a specific period of time), followed by spectroscopic or other analysis of the reaction products.

To determine the level of a marker such as a polypeptide descried herein e.g., SEQ ID NO:01-49, the signal detected from the reporter group that remains bound to the solid support is generally compared to a signal that corresponds to a predetermined cut-off value. In one embodiment, the cut-off value for the detection of a cancer is the average mean signal obtained when the immobilized antibody is incubated with samples from patients without the cancer. In general, a sample generating a signal that is three standard deviations above or below the predetermined cut-off value is considered positive for the cancer. For example, an increased level of certain polypeptides descried herein e.g., SEQ ID NO: 1-5, 7-19, 21, 23-45, and 47-48, may be indicative of the presence of cancer or the stage of cancer. Similarly, a reduced level of certain polypeptides descried herein may e.g., SEQ ID NO: 6, 20, 22, and 46, be indicative of the presence of cancer or the stage of cancer. In some embodiments, the cut-off value is determined using a Receiver Operator Curve, according to the method of Sackett et al., Clinical Epidemiology: A Basic Science for Clinical Medicine, Little Brown and Co., 1985, p. 106-7. Briefly, in this embodiment, the cut-off value may be determined from a plot of pairs of true positive rates (i.e., sensitivity) and false positive rates (100%-specificity) that correspond to each possible cut-off value for the diagnostic test result. The cut-off value on the plot that is the closest to the upper left-hand corner (i.e., the value that encloses the largest area) is the most accurate cut-off value, and a sample generating a signal that is higher than the cut-off value determined by this method may be considered positive. Alternatively, the cut-off value may be shifted to the left along the plot, to minimize the false positive rate, or to the right, to minimize the false negative rate.

In a related embodiment, the assay is performed in a flow-through or test strip format, wherein the binding agent is immobilized on a membrane, such as nitrocellulose. In the flow-through test, target polypeptides within the sample bind to the immobilized binding agent as the sample passes through the membrane. A second, labeled binding agent then binds to the binding agent-polypeptide complex as a solution containing the second binding agent flows through the membrane. The detection of bound second binding agent may then be performed as described herein. In the test strip format, one end of the membrane to which binding agent is hound is immersed in a solution containing the sample. The sample migrates along the membrane through a region containing second binding agent and to the area of immobilized binding agent. The amount of immobilized antibody indicates the presence, or absence or progression or stage of a cancer. Typically, the concentration of second binding agent at that site generates a pattern, such as a line, that can be read visually. In general, the amount of binding agent immobilized on the membrane is selected to generate a visually discernible pattern when the biological sample contains a level of polypeptide that would be sufficient to generate a positive signal in the two-antibody sandwich assay, in the format discussed above. Preferred binding agents for use in such assays are antibodies and antigen-binding fragments thereof. Preferably, the amount of antibody immobilized on the membrane ranges from about 25 ng to about 1 μg, and more preferably from about 50 ng to about 500 ng. Such tests can typically be performed with a very small amount of biological sample.

Quantitative Western blotting also can be used to detect a target molecule or to determine a level of target molecule in a method provided herein. Western blots can be quantitated by well known methods such as scanning densitometry. As an example, protein samples are electrophoresed on 10% SDS-PAGE Laemmli gels. Primary murine monoclonal antibodies, for example, against a target molecule are reacted with the blot, and antibody binding confirmed to be linear using a preliminary slot blot experiment. Goat anti-mouse horseradish peroxidase-coupled antibodies (BioRad) are used as the secondary antibody, and signal detection performed using chemiluminescence, for example, with the Renaissance chemiluminescence kit (New England Nuclear; Boston, Mass.) according to the manufacturer's instructions. Autoradiographs of the blots are analyzed using a scanning densitometer (Molecular Dynamics; Sunnyvale, Calif.) and normalized to a positive control. Values are reported, for example, as a ratio between the actual value to the positive control (densitometric index). Such methods are well known in the art as described, for example, in Parra et al., J. Vase. Surg. 28:669-675 (1998), incorporated herein by reference in its entirety.

As described herein, immunoassays including, for example, enzyme-linked immunosorbent assays, radioimmunoassays and quantitative western analysis, can be useful in some embodiments for detecting a target molecule or determining a level of a target molecule. Such assays typically rely on one or more antibodies. As would be understood by the skilled artisan, methods described herein can be used to readily distinguish proteins with alternative forms of post-translation modifications, e.g., phosphorylated proteins, and glycosylated proteins.

Some embodiments of the methods and compositions provided herein include generating agents that selectively bind to target molecules. In some embodiments, such agents include an antibody or antigen-binding fragment thereof. Methods of generating polyclonal antibodies and monoclonal antibodies are well known in the art. The antibodies or active fragments thereof may be obtained by methods known in the art for production of antibodies or functional portions thereof. Such methods include, but are not limited to, separating B cells with cell-surface antibodies of the desired specificity, cloning the DNA expressing the variable regions of the light and heavy chains and expressing the recombinant genes in a suitable host cell. Standard monoclonal antibody generation techniques can be used wherein the antibodies are obtained from immortalized antibody-producing hybridoma cells. These hybridomas can be produced by immunizing animals with HSCs or progeny thereof, and fusing B lymphocytes from the immunized animals, preferably isolated from the immunized host spleen, with compatible immortalized cells, preferably a B cell myeloma.

In embodiments where the target molecule is a polypeptide associated with one or more iron atoms, antibodies which differentially bind to the iron-associated polypeptide relative to the same polypeptide without iron can be prepared. Antibodies which differentially bind to metal-associated polypeptides relative to the same polypeptide without metal and methods for making such antibodies have been described, for example, in HALLAB, et al., In vitro Reactivity to Implant Metals Demonstrates a Person Dependent Association with both T-Cell and B-Cell Activation, J. Biomed Mater Res A, 2010 February; 92(2):667-682; KONG, et al., Preparation of specific monoclonal antibodies against chelated copper ions, Biol Trace Elem Res., 2012 March; 145(3):388-395; LIU, et al., Preparation and characterization of monoclonal antibody specific for copper-chelate complex, J Immunol Methods., 2013 Jan. 31; 387(1-2):228-236; XIANG. et al., A competitive indirect enzyme-linked immunoassay for lead ion measurement using mAbs against the lead-DTPA complex, Environ Pollut., 2010 May; 158(5):1376-1380; YANG, et al., Detection of antibodies against corrosion products in patients after Co—Cr total joint replacements, J Biomed Mater Res., 1994 Nov.; 28(11):1249-1258; ZHANG, et al., Development of ELISA for detection of mercury based on specific monoclonal antibodies against mercury-chelate, Biol Trace Elem Res., 2011 December; 144(1-3):854-864; and ZHU, et al., Preparation of specific monoclonal antibodies (MAbs) against heavy metals: MAbs that recognize chelated cadmium ions, J Agric Food Chem., 2007 Sep. 19; 55(19):7648-7653, each of which is incorporated by reference in its entirety.

Target molecules, such as protein target molecules, can be characterized, isolated, purified, or obtained for use in generating antibodies by a variety of methods. Proteins, polypeptides and peptides can be isolated by a variety of methods well known in the art, such as protein precipitation, chromatography (e.g., reverse phase chromatography, size exclusion chromatography, ion exchange chromatography, liquid chromatography), affinity capture, and differential extractions.

Isolated proteins can under go enzymatic digestion or chemical cleavage to yield polypeptide fragments and peptides. Such fragments can be identified and quantified. A particularly useful method for analysis of polypeptide/peptide fragments and other target molecules is mass spectrometry (U.S. Pat. App. No. 20100279382, incorporated by reference in its entirety). A number of mass spectrometry-based quantitative proteomics methods have been developed that identify the proteins contained in each sample and determine the relative abundance of each identified protein across samples (Flory et al., Trends Biotechnol. 20:523-29 (2002); Aebersold, J. Am. Soc. Mass Spectrom. 14:685-695 (2003); Aebersold, J. Infect. Dis. 187 Suppl 2:S315-320 (2003); Patterson and Aebersold, Nat. Genet. 33 Suppl, 311-323 (2003); Aebersold and Mann, Nature 422:198-207 (2003); Aebersold, R. and Cravatt, Trends Biotechnol. 20:S1-2 (2002); Aebersold and Goodlett, Chem. Rev. 101, 269-295 (2001); Tao and Aebersold, Curr. Opin. Biotechnol. 14:110-118 (2003), each incorporated by reference in its entirety). Generally, the proteins in each sample are labeled to acquire an isotopic signature that identifies their sample of origin and provides the basis for accurate mass spectrometric quantification. Samples with different isotopic signatures are then combined and analyzed, typically by multidimensional chromatography tandem mass spectrometry. The resulting collision induced dissociation (CID) spectra are then assigned to peptide sequences and the relative abundance of each detected protein in each sample is calculated based on the relative signal intensities for the differentially isotopically labeled peptides of identical sequence.

More techniques for identifying and quantifying target molecules include label-free quantitative proteomics methods. Such methods include: (i) sample preparation including protein extraction, reduction, alkylation, and digestion; (ii) sample separation by liquid chromatography (LC or LC/LC) and analysis by MS/MS; (iii) data analysis including peptide/protein identification, quantification, and statistical analysis. Each sample can be separately prepared, then subjected to individual LC-MS/MS or LC/LC-MS/MS runs (Zhu W. et al., J. of Biomedicine and Biotech. (2010) Article ID 840518, 6 pages, incorporated by reference in its entirety). An exemplary technique includes LC-MS in which the mass of a peptide coupled with its corresponding chromatographic elution time as peptide properties that uniquely define a peptide sequence, a method termed the accurate mass and time (AMT) tag approach. Using LC coupled with Fourier transform ion cyclotron resonance (LC-FTICR) MS to obtain the chromatographic and high mass accuracy information, peptide sequences can be identified by matching the AMT tags to previously acquired LC-MS/MS sequence information stored in a database. By taking advantage of the observed linear correlation between peak area of measured peptides and their abundance, these peptides can be relatively quantified by the signal intensity ratio of their corresponding peaks compared between MS runs (Tang, K., et al., (2004) J. Am. Soc. Mass Spectrom. 15:1416-1423; and Chelius, D. and Bondarenko, P. V. (2002) J. Proteome Res. 1: 317-323, incorporated by reference in their entireties). Statistics tools such as the Student's t-test can be used to analyse data from multiple LC-MS runs for each sample (Wiener, M. C., et al., (2004) Anal. Chem. 76:6085-6096, incorporated by reference in its entirety). At each point of acquisition time and m/z, the amplitudes of signal intensities from multiple LC-MS runs can be compared between two samples to detect peptides with statistically significant differences in abundance between samples.

As will be understood, a variety of mass spectrometry systems can be employed in the methods for identifying and/or quantifying a polypeptide/peptide fragments. Mass analyzers with high mass accuracy, high sensitivity and high resolution include, ion trap, triple quadrupole, and time-of-flight, quadrupole time-of-flight mass spectrometeres and Fourier transform ion cyclotron mass analyzers (FT-ICR-MS). Mass spectrometers are typically equipped with matrix-assisted laser desorption (MALDI) or electrospray ionization (ESI) ion sources, although other methods of peptide ionization can also be used. In ion trap MS, analytes are ionized by ESI or MALDI and then put into an ion trap. Trapped ions can then be separately analyzed by MS upon selective release from the ion trap. Fragments can also be generated in the ion trap and analyzed. Sample molecules such as released polypeptide/peptide fragments can be analyzed, for example, by single stage mass spectrometry with a MALDI-TOF or ESI-TOF system. Methods of mass spectrometry analysis are well known to those skilled in the art (see, e.g., Yates, J. (1998) Mass Spect. 33:1-19; Kinter and Sherman, (2000) Protein Sequencing and Identification Using Tandem Mass. Spectrometry, John Wiley & Sons, New York; and Aebersold and Goodlett, (2001) Chem. Rev. 101:269-295, each incorporated by reference in its entirety).

For high resolution polypeptide fragment separation, liquid chromatography ESI-MS/MS or automated LC-MS/MS, which utilizes capillary reverse phase chromatography as the separation method, can be used (Yates et al., Methods Mol. Biol. 112:553-569 (1999), incorporated by reference in its entirety). Data dependent collision-induced dissociation (CID) with dynamic exclusion can also be used as the mass spectrometric method (Goodlett, et al., Anal. Chem. 72:1112-1118 (2000), incorporated by reference in its entirety).

Once a peptide is analyzed by MS/MS, the resulting CID spectrum can be compared to databases for the determination of the identity of the isolated peptide. Methods for protein identification using single peptides have been described previously (Aebersold and Goodlett, Chem. Rev. 101:269-295 (2001); Yates, J. Mass Spec. 33:1-19 (1998), David N. et al., Electrophoresis, 20 3551-67 (1999), each incorporated by reference in its entirety). In particular, it is possible that one or a few peptide fragments can be used to identify a parent polypeptide from which the fragments were derived if the peptides provide a unique signature for the parent polypeptide. Moreover, identification of a single peptide, alone or in combination with knowledge of a site of glycosylation, can be used to identify a parent glycopolypeptide from which the glycopeptide fragments were derived. As will be understood, methods that include MS can be used to characterize proteins, fragments thereof, as well as other types of target molecules described herein.

In some embodiments, target molecules include nucleic acids. Nucleic acids can encode a polypeptide or fragment thereof useful to determine the presence or absence of a cancer. As such, target molecules include nucleic acid molecules sufficient for use as hybridization probes to identify nucleic acid molecules that correspond to a target molecule, including nucleic acids which encode a polypeptide corresponding to a target molecules, and fragments of such nucleic acid molecules, e.g., those suitable for use as PCR primers for the amplification or mutation of nucleic acid molecules. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

A nucleic acid target molecule can be amplified using cDNA, mRNA, or genomic DNA as a template and appropriate oligonucleotide primers according to standard PCR amplification techniques. The nucleic acid so amplified can be cloned into an appropriate vector and characterized by DNA sequence analysis. Furthermore, oligonucleotides corresponding to all or a portion of a nucleic acid target molecule can be prepared by standard synthetic techniques, e.g., using an automated DNA synthesizer.

In another preferred embodiment, a nucleic acid target molecule comprises a nucleic acid molecule that has a nucleotide sequence complementary to a nucleic acid which is differentially expressed in cancer or a fragment thereof. For example, the target molecule may comprise a nucleic acid encoding a polypeptide of any one of SEQ ID NO.s: 1-49 or a fragment comprising at least 10, at least 20, at least 30, at least 40, at least 50 or more consecutive nucleotides thereof. A nucleic acid molecule which is complementary to a given nucleotide sequence is one which is sufficiently complementary to the given nucleotide sequence that it can hybridize to the given nucleotide sequence thereby forming a stable duplex.

In some embodiments, a fragment of a polynucleotide sequence will be understood to include any nucleotide fragment having, for example, at least about 5 successive nucleotides, at least about 12 successive nucleotides, at least about 15 successive nucleotides, at least about 18 successive nucleotides, or at least about 20 successive nucleotides of the sequence from which it is derived. An upper limit for a fragment can include, for example, the total number of nucleotides in a full-length sequence encoding a particular polypeptide. A fragment of a polypeptide sequence will be understood to include any polypeptide fragment having, for example, at least about 5 successive residues, at least about 12 successive residues, at least about 15 successive residues, at least about 18 successive residues, or at least about 20 successive residues of the sequence from which it is derived. An upper limit for a fragment can include, for example, the total number of residues in a full-length sequence of a particular polypeptide.

Moreover, a nucleic acid target molecule can comprise all or only a portion of a nucleic acid sequence which is differentially expressed in cancer. For example, the target molecule may comprise a nucleic acid encoding a polypeptide of SEQ ID NO.s: 1-49 or a fragment comprising at least 10, at least 20, at least 30, at least 40, at least 50 or more consecutive nucleotides thereof. Such nucleic acids can be used, for example, as a probe or primer. The probe/primer typically is used as one or more substantially purified oligonucleotides. The oligonucleotide typically comprises a region of nucleotide sequence that hybridizes under stringent conditions to at least about 7, preferably about 15, more preferably about 25, 50, 75, 100, 125, 150, 175, 200, 250, 300, 350, or 400 or more consecutive nucleotides of a nucleic acid.

Probes based on the sequence of a nucleic acid target molecule can be used to detect transcripts or genomic sequences corresponding to one or more target molecules. The probe comprises a label group attached thereto, e.g., a radioisotope, a fluorescent compound, an enzyme, or an enzyme co-factor. Such probes can be used as part of a diagnostic test kit for identifying a biological sample, such as fluids, cells or tissues, which mis-express the protein, such as by measuring levels of a nucleic acid molecule encoding the protein in a sample of a fluid or cells from a subject, e.g., detecting mRNA levels or determining whether a gene encoding the protein has been mutated or deleted. Embodiments also include nucleic acid target molecules that differ, due to degeneracy of the genetic code, from the nucleotide sequence of nucleic acids encoding a protein that corresponds to a target molecule, and thus encode the same protein.

### Method for Assessing the Presence, Absence, Progression or Stage of a Cancer

Some of the methods and composition provided herein include methods for assessing the presence absence, progression or stage of a cancer in a female subject. Some such embodiments include determining the level of at least one target molecule in a sample from said subject. In some embodiments, the target molecule comprises at least one polypeptide or fragment thereof or at least one nucleic acid encoding the polypeptide. In some embodiments, the polypeptide is selected from any polypeptide provided herein, for example, SEQ ID NO.s:01-49.

In some embodiments, the sample is obtained from the gynecological tract of a subject. The gynecological tract of a subject can include the ovary, oviduct, endometrium, cervix, vagina, and posterior vaginal fornix. The sample can include a fluid originating from the gynecological tract, such as a mucus secretion of the gynecological tract, such as cervico-vaginal fluid. In some embodiments, a sample can include a wash solution obtained from the gynecological tract. In particular embodiments, the sample is obtained from the cervix, the vagina, or the posterior vaginal fornix. In some embodiments, the sample is obtained from a cervical pap specimen. In some embodiments, the sample is substantially free of cells. In some embodiments, the sample is obtained using a method described in U.S. application Ser. No. 12/646,592, entitled “NOVEL MOLECULAR ASSAY AND USES THEREOF”, the disclosure of which is incorporated herein by reference in its entirety.

Some embodiments include determining the level in the sample of at least 2 target molecules, at least 3 target molecules, at least 4 target molecules, at least 5 target molecules, at least 6 target molecules, at least 7 target molecules, at least 8 target molecules, at least 9 target molecules, at least 10 target molecules, at least 11 target molecules, at least 12 target molecules, at least 13 target molecules, at least 14 target molecules, at least 15 target molecules, at least 16 target molecules, at least 17 target molecules, at least 18 target molecules, at least 19 target molecules, or at least 20 target molecules.

Some embodiments also include comparing the level of at least one target molecule in a sample of a subject with the level of the target molecule in a sample from a subject without the cancer. Some embodiments also include comparing the level of at least one target molecule in a sample of a subject with the level of the target molecule in a sample from a subject with the cancer.

In some embodiments, an increase in the level of the target molecule in a sample from a subject compared to the level of the target molecule in a sample from said subject without the cancer is indicative of the presence of the cancer in the subject. In some such embodiments, the target molecule can include a polypeptide or a fragment thereof, a nucleic acid encoding the polypeptide or fragment thereof, in which the polypeptide includes SEQ ID NO.s 1-5, 7-19, 21, 23-45, and 47-48. In some embodiments, the cancer comprises endometrial cancer, and the polypeptide includes SEQ ID NO.s: 1-5, 7-19, 21, and 23-24. In some embodiments, the cancer comprises ovarian cancer, and the polypeptide includes SEQ ID NO.s: 25-45, and 47-48.

In some embodiments, a decrease in the level of the target molecule in a sample from a subject compared to the level of the target molecule in a sample from said subject without the cancer is indicative of the presence of the cancer in the subject. In some such embodiments, the target molecule can include a polypeptide or a fragment thereof, a nucleic acid encoding the polypeptide or fragment thereof, in which the polypeptide includes SEQ ID NO.s 6, 20, 22, and 46. In some embodiments, the cancer comprises endometrial cancer, and the polypeptide includes SEQ ID NO.s: 6, 20, and 22. In some embodiments, the cancer comprises ovarian cancer, and the polypeptide includes SEQ ID NO.s: 46.

In some embodiments, an increase in the level of a target molecule in a sample compared to the level of the target molecule in a sample obtained from a subject without a cancer is indicative of the cancer, in which the increase is at least about a 3-fold increase at least about a 5-fold increase, at least about a 10-fold increase, at least about a 20-fold increase, at least about a 30-fold increase, at least about a 40-fold increase, at least about a 50-fold increase, at least about a 60-fold increase, at least about a 70-fold increase, at least about a 80-fold increase, at least about a 90-fold increase, and at least about a 100-fold increase.

In some embodiments, a decrease in the level of a target molecule in a sample compared to the level of the target molecule in a sample obtained from a subject without a cancer is indicative of the cancer, in which the decrease is at least about a 3-fold decrease at least about a 5-fold decrease, at least about a 10-fold decrease, at least about a 20-fold decrease, at least about a 30-fold decrease, at least about a 40-fold decrease, at least about a 50-fold decrease, at least about a 60-fold decrease, at least about a 70-fold decrease, at least about a 80-fold decrease, at least about a 90-fold decrease, and at least about a 100-fold decrease.

Methods to determine the level of a target molecule in a sample are well known in art. Some examples of such methods are provided herein. In some embodiments, a method for determining the level of a target molecule, such as a polypeptide or fragment thereof, can include an immunoassay. Examples of an immunoassay include a Western blot, an enzyme linked immunoabsorbent assay (ELISA), and radioimmunoassay. In some embodiments, a method for determining the level of a target molecule, such as a polypeptide or fragment thereof, can include mass spectrometry.

In some embodiments, the cancer is a non-cervical cancer of the gynecological tract. Examples of such cancers include endometrial cancer and ovarian cancer. As used herein, the term “endometrial cancer” refers to, but is not limited to endometrial carcinomas and endometrial adenocarcinomas. Endometrial cancers as used herein also include other well-known cell types such as papillary serous carcinoma, clear cell carcinoma, papillary endometrioid carcinoma, and mucinous carcinoma. Endometrial cancers also include endometrial hyperplasia, endometrial hyperplasia with atypia, and non-invasive endometrial cancer. As used herein, the term “ovarian cancer” refers to, but is not limited to ovarian tumors, carcinomas, (e.g., carcinoma in situ, invasive carcinoma, metastatic carcinoma) and pre-malignant conditions. By “ovarian tumor” is meant both benign and malignant tumors, such as ovarian germ cell tumors, e.g. teratomas, dysgerminoma, endodermal sinus tumor and embryonal carcinoma, and ovarian stromal tumors, e.g. granulosa, theca, Sertoli, Leydig, and collagen-producing stromal cells. Ovarian cancers as used herein also include art recognized histological tumor types, which include, for example, serous, mucinous, endometrioid, and clear cell tumors. The term ovarian cancer as used herein further includes art recognized grade and stage scales: grade I, II and III and stage I (including stage IA, IB and IC), II (including stage IIA, IIB and IIC), III (including stage IIIA, IIIB and IIIC), and IV.

In some embodiments, the subject is mammalian, for example, human.

### Kits

Some embodiments include a kit for determining the presence or absence or stage of a cancer in a female subject. In some such embodiments, the kit can include (a) a suitable diluent for irrigating the uterine cavity of the subject; (b) a receptacle for collection of the diluted uterine fluid; and (c) an agent that selectively binds to at least one target molecule. In some embodiments, the target molecule comprises a polypeptide or fragment thereof, or a nucleic acid encoding a polypeptide or fragment thereof. In some such embodiments, the polypeptide includes SEQ ID NO.s:01-48 and 49.

Some kits include at least three agents that each selectively bind to a different target molecule, such as a polypeptide or a nucleic acid encoding said polypeptide. Some kits include at least five agents that each selectively bind to a different target molecule, such as a polypeptide or a nucleic acid encoding said polypeptide. Some kits include at least ten agents that each selectively bind to a different target molecule, such as a polypeptide or a nucleic acid encoding said polypeptide. In some embodiments, the agent comprises an antibody or antigen-binding fragment thereof.

In some embodiments, a kit comprises a molecule which selectively binds to a polypeptide comprising a sequence selected from the group consisting of a polypeptide comprising, consisting essentially of, or consisting of SEQ ID NO.s: 1-49. or a nucleic acid encoding a polypeptide, such as a polypeptide selected from SEQ ID NO.s: 1-49, affixed to a solid support. In some embodiments, a kit comprises a plurality of molecules which selectively binds to a polypeptide comprising a sequence selected from the group consisting of a polypeptide comprising, consisting essentially of, or consisting of SEQ ID NO.s: 1-49. or a nucleic acid encoding a polypeptide, such as a polypeptide selected from SEQ ID NO.s: 1-49, affixed to a solid support. In some embodiments, a kit can also include a detectable agent which selectively binds to a target molecule.

Some embodiments include a kit comprising an agent which selectively binds to at least one polypeptide comprising an amino acid sequence selected from the group consisting of a polypeptide comprising, consisting essentially of, or consisting of SEQ ID NO.s:01-49 or a fragment thereof, wherein said agent is attached to a solid support. In some embodiments, a plurality of agents that bind to different polypeptides comprising an amino acid sequence selected from the group consisting of a polypeptide comprising, consisting essentially of, or consisting of SEQ ID NO.s:01-49 or a fragment thereof are attached to said solid support. In some embodiments, the solid support comprises a solid phase test strip. Some embodiments also include a detectable agent which selectively binds to said polypeptide.

Some embodiments include a kit comprising an agent which selectively binds to at least one nucleic acid encoding a polypeptide comprising an amino acid sequence selected from the group consisting of a polypeptide comprising, consisting essentially of, or consisting of SEQ ID NO.s:01-49 or a fragment thereof, wherein said agent is attached to a solid support. In some embodiments, a plurality of agents that bind to nucleic acids encoding different polypeptides comprising an amino acid sequence selected from the group consisting of a polypeptide comprising, consisting essentially of, or consisting of SEQ ID NO.s:01-49 or a fragment thereof are attached to said solid support. In some embodiments, the solid support comprises a solid phase test strip. Some embodiments also include a detectable agent which selectively binds to said polypeptide.

Some embodiments of the methods and compositions provided herein include isolated polypeptides consisting essentially of an amino acid sequence selected from the group consisting of a polypeptide comprising, consisting essentially of, or consisting of SEQ ID NO.s:01-49 or a fragment thereof, wherein said polypeptide is differentially expressed in cancer. Some embodiments of the methods and compositions provided herein include isolated polypeptides consisting of an amino acid sequence selected from the group consisting of a polypeptide comprising, consisting essentially of, or consisting of SEQ ID NO.s:01-49 or a fragment thereof, wherein said polypeptide is differentially expressed in cancer.

Some embodiments of the methods and compositions provided herein include isolated nucleic acids encoding a polypeptide consisting essentially of an amino acid sequence selected from the group consisting of a polypeptide comprising, consisting essentially of, or consisting of SEQ ID NO.s:01-49 or a fragment thereof, wherein said polypeptide is differentially expressed in cancer. Some embodiments of the methods and compositions provided herein include isolated nucleic acids encoding a polypeptide consisting of an amino acid sequence selected from the group consisting of a polypeptide comprising, consisting essentially of, or consisting of SEQ ID NO.s:01-49 or a fragment thereof, wherein said polypeptide is differentially expressed in cancer.

Some embodiments of the methods and compositions provided herein include isolated agents that selectively bind to an isolated polypeptide consisting of an amino acid sequence selected from the group consisting of a polypeptide comprising, consisting essentially of, or consisting of SEQ ID NO.s:01-49 or a fragment thereof, wherein said polypeptide is differentially expressed in cancer. In some such embodiments, the agent comprises an antibody or antigen-binding fragment thereof.

### EXAMPLES

### Example 1

### Sample Collection

IRB approval was obtained (USA IRB #09-034 3/412009) according to institutional procedures for collection of cervico-vaginal secretions. Patients gave signed written consent to have these samples collected during routine pelvic examinations within the University of South Alabama (USA) and Mobile Infirmary Health System (MIMC) facilities. Samples were collected within the clinic space as well as operating rooms at each hospital. Patients aged 21 or older at time of informed consent who had a uterus were eligible for sample collection. Patients with prior hysterectomy, lack of clinical data, or lack of follow-up were excluded from this study. Physicians involved within the study collected data from chart review of clinic notes, operative reports, pathology reports and entered this information into a password protected centralized computerized database. Patients were initially categorized into categories based on information available at their initial presentation. These categories were broad and were further refined once final pathology was available. For example, a patient might initially be categorized as having an “ovarian cyst/pelvic mass”. This would be entered into the database as the primary diagnosis. If, after having surgery, she was found to have ovarian cancer, endometriosis, and fibroids, all of these diagnoses would have been entered as the final diagnoses. Patients were grouped into more specific diagnostic categories based on their final histologic diagnosis such as: Endometrial cancer, Ovarian cancer, Endometriosis, Uterine Fibroids, Infertility, Pregnancy, benign pelvic mass, etc. These groups were subdivided into “pure” and “mixed” samples based on the absence or presence of alternative confounding diagnoses. Data variables included patient demographics, surgicopathologic data, cancer related data, and comorbid conditions. All clinical data was stripped of patient identifiers and coded with patient study number, sample number by the data coordinator and keep in a separate location. Researchers involved in the basic science aspect of the data analysis were blinded to patient identifiers. Samples were collected by IRB approved physicians within the USA and MIMC health system gynecologic clinics and/or within the operating room after anesthesia induction and prior to surgery. First, a dacron tipped swab was placed in the vaginal vault for approximately 15 seconds and then immediately placed within a preservative solution for storage/transport and labeled with sample number and code for vaginal sample. Second, a standard cytobrush was placed within the cervical os (in the endocervical canal) and turned several times, (identical to Pap smear techniques) and also placed within a preservative solution for storage/transport and labeled with sample number and code for cervical sample. For each patient in the study, a vaginal and cervical sample was obtained in both the clinic setting as well as the operating room setting for those who were undergoing surgery. For a small selected group of patients, a tampon collection was obtained. The patient was given a study tampon and was instructed to insert “x” hours before surgery/clinic. The tampon was removed by the physician and placed in the preservative solution as described herein. Other volunteers representing healthy controls with no gynecological diseases were also provided with tampons, and the volunteer placed the tampon into the vagina in the normal way and removed it after “x” minutes and placed into the provided liquid.

Coded samples were collected from clinic on a daily basis and logged into proteomic laboratory upon arrival. The liquid solution was further processed by centrifugation to remove all or substantially all of the cells and other debris so that the polypeptide analyses described herein involves the soluble proteins contained in the liquid solution; the cells and pellet were discarded. The resulting fluid was stored at −80 C until analysis. Proteins were isolated from the samples by dispersing approximately 1% of the sample into 0.1% trifluoroacetic acid (TFA). Proteins were eluted with 60% Acetonitrile (ACN) on an Agilent C3 pre-column using 2% ACN. Following overnight digestion with trypsin, the samples underwent a triplicate injection into a LTQ-Orbitrap MS with the injection volume based on the UV peak height from the chromatogram. The MS ran on one second scans (peptide mass data collected) with 5 per second MS/MS scans of selected peptide masses. Search files were combined and one large search was done for endometriosis patients versus normal controls. Individuals were then compared via their peptide sequence data using MASCOT search comparisons or DifProWare.

### Example 2

### Identification of Polypeptides Associated with Endometrial Cancer Identification of Polypeptides

Data were acquired on an LTQ-Orbitrap mass spectrometer using input from an LC system. The A solvent contained 3% of B and 0.2% formic acid in water. The B solvent contained 3% of A and 0.2% formic acid in acetonitrile. Solvents were HPLC grade from Fisher. For a 120 min run, the starting solvent was 5% B and remains for 7 min. The gradient was changed to 10% by 13 min, 40% by 83 min, 90% by 103 min, then reduced from 90% to 5% at 111 min. It was then re-equilibrated for the next injection. Three injections were performed for each sample for repeatability determination.

The MS was scanned (Orbitrap) over the mass range from 400 m/z to 2000 m/z every second while the LTQ (Trap) acquired up to 5 MSMS (peptide sequence) spectra in parallel. Data were acquired using the standard Thermo Xcalibur software. MS data (Orbitrap) was stable to 2-3 ppm and a background ion was used for mass drift assessment. MSMS data (LTQ) was measured to approximately 0.6 Da but the parent mass was acquired from the low ppm Orbitrap data. Peptides were eluted from a C18 LC column using triplicate injections to ensure reliability and repeatability of the data. A search file was created from the triplicate injections from each lavage preparation (patient sample) and converted into a MGF (MASCOT Generic Format) file using a combination of Xcalibur and MASCOT software packages.

Database searching was done using the MASCOT search engine (Matrix Science, UK) against the RefSeq database (http://www.ncbi.nlm.nih.gov/RefSeq/) with taxonomy specified as human (homo sapiens), a mass accuracy of 10 ppm for the parent ion (MS) and 0.6 Da for the fragment ions (MS/MS), and “no enzyme” selected. Searching without enzyme specificity was performed due to the presence of digestive enzymes in the sample that may modify or truncate peptides being examined. The RefSeq database was supplemented by the addition of antibody sequences that are included in the SwissProt protein database, as these antibody sequences are not part of the standard RefSeq listing.

Higher MASCOT scores indicated better proteins hits and were correlated to relative protein levels. A score threshold of “>40” was indicative of a p-value significance of <0.05 as determined by the MASCOT scoring system based on the search of this database with no enzyme specificity; a score of 40 is consistent with a p<0.01. Standard MASCOT scoring was used whereby only the highest score was added for each peptide detected, even if it was sampled during MS/MS multiple times. For all data included, scores were all >40 in at least one sample per protein line. For additional confidence, the numbers of significant peptides were also reported and a minimum criterion of at least 2 peptides was selected. Very few had less than 3 peptides. All significant peptides counted represented different sequences (individual peptides) from their respective proteins. The score and numbers of significant peptides are reported in the format x/y where x is the score and y the number of significant peptides. Proteins were reported as protein name and the “gi” number defined by the protein database of the NCBI. The sequences contained in each of the “gi” numbers in the NCBI database listed throughout the present application are incorporated herein by reference. Where a protein is named in its preprotein or other non-mature form, the mature form of the protein is equally implied including such changes as removal of signal sequences and the addition of post-translational modifications. Proteins were named by gene derived sequence to provide consistency.

Identification of Polypeptides Associated with Endometrial Cancer

Sample polypeptide data was derived from 306 LC-MS runs from 102 subjects which included 52 Endometrial Cancer (EmCa) patients and 50 normal control subjects. Subject groups were compared to identify promising candidate markers from among 3740 peptides. After normalization and combining replicate runs from each subject, AUC, Wilcoxon rank sum test were computed to evaluate distributional differences between cancer and normal groups. The Wilcoxon test combining with AUC identified 32 peptides exceeding the 5% false discovery rate (FDR) threshold and AUC 0.80. The Wilcoxon procedure was also performed using non-normalized data to assess the effect of the normalization procedure. In this setting, 10 peptides were identified that exceeded the 5% FDR threshold and AUC 0.80.

### Data Analysis Approach

Endometrial cancer data was analyzed using the Wilcoxon rank-sum test, Fisher's exact test, fold change, and a ROC curve analysis to identify potentially useful biomarkers. A false discovery rate method was applied to adjust p-values for multiple comparison.

### Combining Data and Peptide Selection

Endometrial cancer patients data sets were combined. There were a total of 102 (control: 50, disease: 52) subject samples included in the new data and 42 in old data set each with 3 runs. Among the subjects in old data, 11 subjects were not in new data. Among the disease subjects, 28 had co-existing diagnoses and 24 without co-existing diagnoses. In the new data set there were 6 disease subject samples from surgical patients which were also included in this analysis. After removal of duplicates (multiple MASCOT matches), the new data contain 3740 peptide bins for 306 LC-MS runs (samples from the cervix of patients in the clinic). The old data contain 3931 peptide bins. The samples were grouped into 3 non-disjoint sets for analysis: (1) Old subjects: Subjects in old data set; (2) All subjects: Subjects in new data; and (3) New subjects: Subjects in new data but not in old data. Peptide signals were screened as follows: (1) For the old data set, we identify peptide signals were identified that met the 5% FDR adjusted Wilcoxon test p-value. Seventy (70) signals met this criterion. (2. For the new data set with all subjects, 2615 peptide signals exceeded the 0.05 FDR threshold. This large number was filtered, and only those signals with AUC greater than 0.80 and Wilcoxon test FDR p-value less than 0.01 were selected. (3) For NEW subjects in current data set, 2400 peptide signals exceeded the 0.05 FDR threshold. As above, only those signals with AUC greater 0.80 and Wilcoxon test FDR p-value less than 0.01 were filtered and retained. This still resulted in 115 signals.

### LASSO Logistic Regression

Using the Wilcoxon test P-values, the first 100 peptides with smallest p-values as candidate predictors in a classification model were selected for further analysis. These peptide predictors to fit a logistic regression model to classify each subject's disease status. A statistical method known as the Lasso was used to screen potential predictors (Tibshirani, R. (1996). “Regression Shrinkage and Selection via the Lasso” J. Roy. Statist. Soc. Ser. B., 58 (1): 267-288, incorporated by reference in its entirety). Table 1, Table 2, and Table 3 summarize peptides from groups (1) Old subjects, (2) All subjects, and (3) New subjects, respectively, which were further selected using logistic regression through a LASSO selection model.

TABLE 1

| Mass | Time |
|---|---|
| 922.14 | 67.79 |
| 1016.573 | 33.75 |
| 1041.58 | 32.29 |
| 1383.698 | 43.97 |
| 1860.615 | 69.42 |
| 2384.165 | 36.07 |
| 4318.333 | 68.3 |

TABLE 2

| Mass | Time |
|---|---|
| 1212.675 | 69.38 |
| 1431.608 | 29.81 |
| 1534.741 | 41.64 |
| 2097.006 | 52.97 |
| 2996.461 | 53.07 |
| 2996.479 | 60.83 |
| 3304.741 | 52.19 |

TABLE 3

| Mass | Time |
|---|---|
| 561.775 | 36.97 |
| 1041.58 | 32.65 |
| 1066.509 | 41.81 |
| 1212.675 | 69.38 |
| 1251.611 | 33.86 |
| 1385.708 | 61.71 |
| 1431.608 | 29.81 |
| 2098.006 | 52.99 |
| 5673.911 | 70.26 |

### Correlation Between Peptides

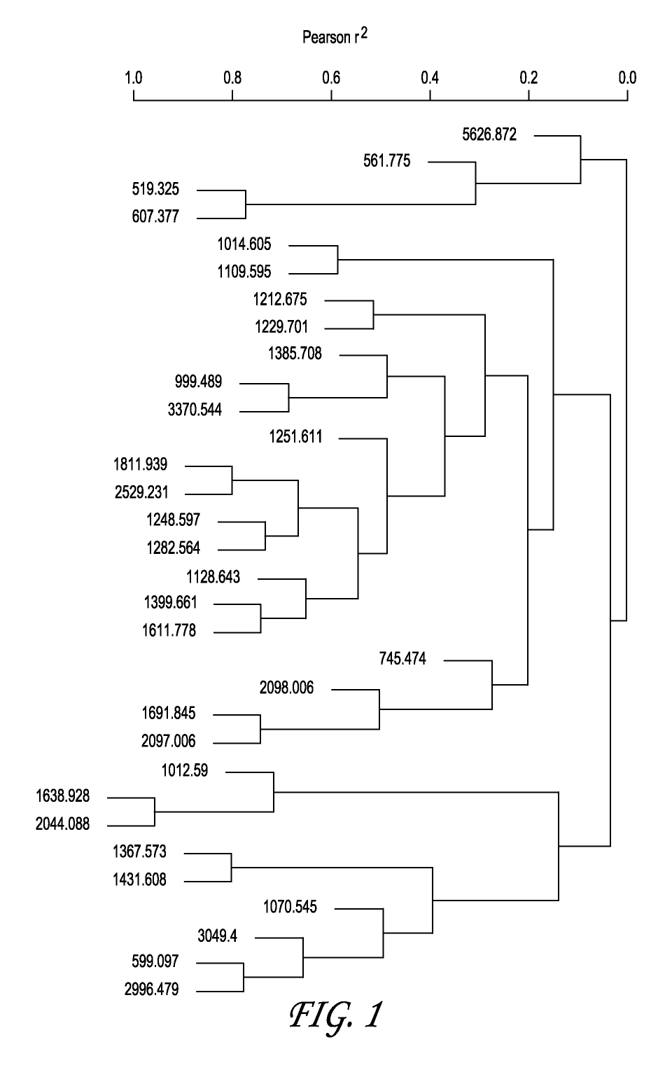

The correlation between certain peptide signals was investigated using an cluster analysis. The cluster analysis of 32 selected peptides by Wilcoxon test FDR p-value 0.05 and AUC greater than 0.80 is shown in FIG. 1. The peptides clusters indicate signals that rise and fall together (across samples). Note that signal groups that were relatively uncorrelated were combined to provide approximately independent information in a screening or diagnostic panel.

Table 4 summarizes the results for polypeptides identified and associated with endometrial cancer.

TABLE 4

| | | | | Relative abundance | | | | |
|---|---|---|---|---|---|---|---|---|
| | | | Ions | | | Patient/ | | SEQ |
| Protein ID | Mass | Time | score | Patient | Control | Control | Peptide sequence | ID NO |
| gi\|4502027: albumin | 2098.006 | 52.99 | 63 | 326795 | 23125 | 14.13 | VFDEFKPLVEEPQNLIK | 01 |
| 397-413 | | | | | | | | |
| gi\|4502027: albumin | 1012.59 | 42.11 | 107 | 2844101 | 857464 | 3.317 | LVAASQAALGL | 02 |
| preproprotein | | | | | | | | |
| gi\|4502027: albumin | 2044.088 | 53 | 95 | 9857009 | 3E+06 | 3.351 | VFDEFKPLVEEPQNLIK | 03 |
| preproprotein | | | | | | | | |
| gi\|4502027: albumin | 1638.928 | 35.12 | 107 | 6924100 | 2E+06 | 3.194 | KVPQVSTPTLVEVSR | 04 |
| preproprotein | | | | | | | | |
| gi\|4502027: albumin | 1066.509 | 41.81 | 64 | 22423 | 1620 | 13.84 | LVAASQAALGL | 05 |
| 599-609 | | | | | | | | |
| glycopeptide #1 | 3370.544 | 22.39 | | 77057 | 17806 | 4.327 | | |
| gi\|21536452 mesotrypsin | 1534.741 | 41.64 | 91 | 2284 | 34169 | 0.067 | SLPYQVSLNSGSHF | 06 |
| isoform 2 preproprotein | | | | | | | | |
| non-tryptic | | | | | | | | |
| gi\|4557321: | 1282.564 | 36.55 | 60 | 76960 | 6243 | 12.33 | WQEEMELYR | 07 |
| apolipoprotein A-I | | | | | | | | |
| preproprotein | | | | | | | | |
| gi\|4557321: | 1385.708 | 61.71 | 109 | 195822 | 27885 | 7.022 | VSFLSALEEYTK | 08 |
| apolipoprotein A-I | | | | | | | | |
| preproprotein | | | | | | | | |
| gi\|4557321: | 1611.778 | 49.45 | 109 | 136635 | 14137 | 9.665 | LLDNWDSVTSTFSK | 09 |
| apolipoprotein A-I | | | | | | | | |
| preproprotein | | | | | | | | |
| gi\|4557871: transferrin | 2529.231 | 68.73 | 72 | 79690 | 23677 | 3.366 | SMGGKEDLIWELLNQAQEHFGK | 11 |
| gi\|4557321: | 1399.661 | 40.52 | 121 | 53242 | 5429 | 9.808 | DYVSQFEGSALGK | 12 |
| apolipoprotein A-I | | | | | | | | |
| preproprotein | | | | | | | | |
| gi\|4557871: transferrin | 1248.597 | 37.58 | 83 | 81296 | 13965 | 5.821 | SASDLTWDNLK | 13 |
| gi\|4557321: | 1229.701 | 59.01 | 57 | 178829 | 21298 | 8.397 | QGLLPVLESFK | 14 |
| apolipoprotein A-I | | | | | | | | |
| preproprotein | | | | | | | | |
| glycopeptide #1 | 3661.638 | 22.75 | | 6717 | 445 | 15.09 | | |
| alpha-1b-glycoprotein | 1236.638 | 51.65 | 52 | 21387 | 3423 | 6.249 | R.LETPDFQLFK.N | 15 |
| gi\|11321561: hemopexin | 1128.643 | 55.71 | 50 | 55130 | 9712 | 5.677 | RLWWLDLK | 16 |
| precursor | | | | | | | | |
| gi\|4504345: alpha 2 | 1070.545 | 47.33 | 60 | 1.3E+07 | 4E+06 | 3.1 | MFLSFPTTK | 17 |
| globin | | | | | | | | |
| gi\|50363217: serine | 1014.605 | 41.43 | 80 | 238779 | 44382 | 5.38 | SVLGQLGITK | 18 |
| proteinase inhibitor: | | | | | | | | |
| clade A: member 1 | | | | | | | | |
| gi\|4504345: alpha- | 3049.4 | 59.55 | 57 | 225952 | 48202 | 4.688 | VADALTNAVAHVDDMPNALSAL | 19 |
| globin 2995.481 Fmod | | | | | | | SDLHAHK | |

| | | | | | | | | |
|---|---|---|---|---|---|---|---|---|
| gi\|155969697: keratin | 1114.585 | 36.58 | 63 | 6794 | 59756 | 0.114 | LEGLEDALQK | 20 |
| 6C | | | | | | | | |
| gi\|50363217: serine | 1109.595 | 36.48 | 66 | 210869 | 42278 | 4.988 | LSITGTYDLK | 21 |
| proteinase inhibitor: | | | | | | | | |
| clade A: member 1 | | | | | | | | |
| gi\|4506145: protease: | 2226.09 | 39.88 | 56 | 13037 | 84604 | 0.154 | LGEHNIEVLEGNEQFINAAK | 22 |
| serine: 1 preproprotein | | | | | | | | |
| gi\|4557321: | 1212.675 | 69.38 | 61 | 27693 | 3105 | 8.919 | QGLLPVLESFK | 23 |
| apolipoprotein A-I | | | | | | | | |
| preproprotein | | | | | | | | |
| gi\|4504345: alpha 2 | 2996.461 | 53.07 | 124 | 403871 | 89637 | 4.506 | VADALTNAVAHVDDMPNALSAL | 24 |
| globin | | | | | | | SDLHAHK | |

### Example 3

### Identification of Polypeptides Associated with Ovarian Cancer Identification of Polypeptides

Candidate polypeptides were identified from samples by mass spectrometry as described in Example 2.

Identification of Polypeptides Associated with Ovarian Cancer

Sample peptides from 249 LC-MS runs from 83 subjects which included 33 ovarian cancer (OVCA) patients and 50 normal control subjects were evaluated. Biomarker study subject groups were compared to identify promising candidate markers among 2942 peptides. After normalization and combining replicate runs from each subject, AUC, Wilcoxon rank sum test were computed to evaluate distributional differences between cancer and normal groups. The Wilcoxon test identified 357 peptides exceeding the 5% false discovery rate (FDR) threshold. The Wilcoxon procedure was also performed using non-normalized data to assess the effect of the normalization procedure. In this setting, 429 peptides were identified that exceeded the 5% FDR threshold. The peptide lists for normalized and non-normalized data contained 298 common peptides.

### Data Analysis Approach

Ovarian cancer data was analyzed using the Wilcoxon rank-sum test, Fisher's exact test, fold change, and a ROC curve analysis to identify potentially useful biomarkers. A false discovery rate method was applied to adjust p-values for multiple comparisons.

### Combination of Data and Peptide Selection

Ovarian cancer patients data sets were combined. There were a total of 83 (control: 50, disease: 33) subject samples included in a new data and 35 subject samples in old data set each with 3 runs. After removal of duplicate rows (multiple MASCOT matches), the new data contain 2942 peptide bins for 249 LC-MS runs (samples from the cervix of patients in the clinic). The old data contained 5129 peptide bins. The samples were grouped into 3 non-disjoint sets for analysis: (1) Old subjects: Subjects in old data; (2) All subjects: Subjects in new data; and (3) New subjects: Subjects in new data but not in old data. Peptide signals were screened as follows: (1). For the old data set, peptide signals were identified that met the 5% FDR adjusted Wilcoxon test p-value. One hundred twenty seven (127) signals met this criterion. These corresponded to those signals identified previously. Further filtering using AUC greater than 0.75, identified 64 peptide signals. (2) For the new data set with all subjects, 357 peptide signals exceeded the 0.05 FDR threshold. As in old data, further filtering using AUC greater than 0.75, 12 peptide signals were identified. (3) For NEW subjects in the data set, 304 peptide signals exceeded the 0.05 FDR threshold. Here we filtered to retain only those signals with AUC greater 0.75 were filtered, this resulted in 62 signals. Table 3

Lists the Top 50 of these Peptide Signals. Correlation Between Peptides

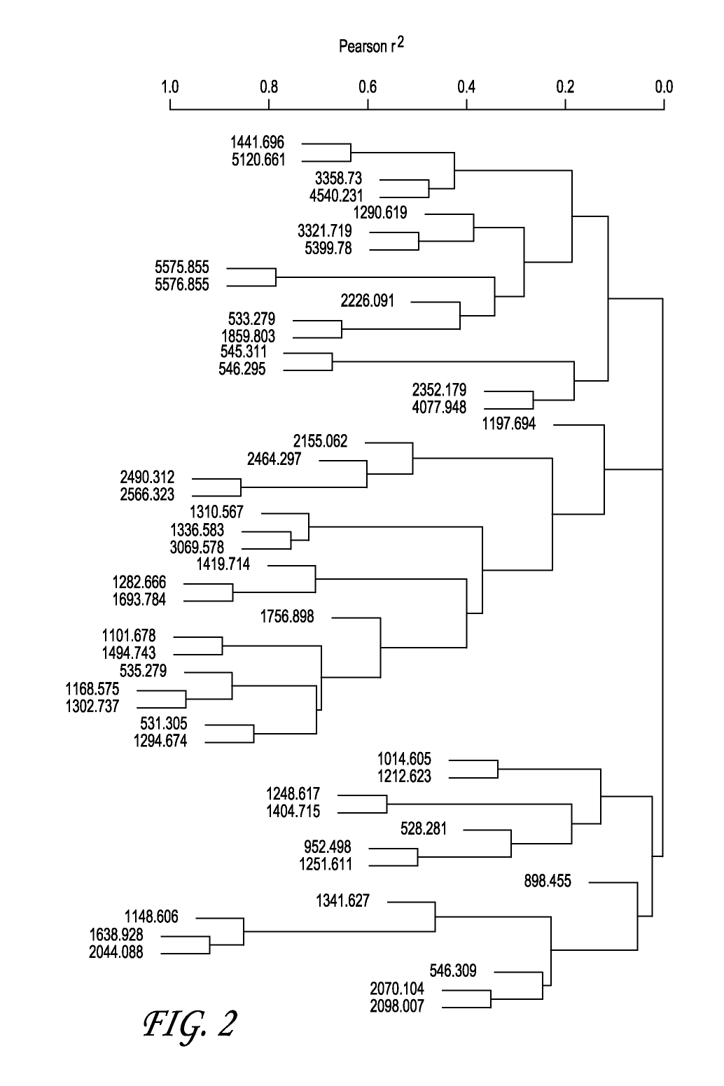

The correlation between certain peptide signals was investigated using cluster analysis. A cluster analysis of first 50 selected peptides (all subjects data) by Wilcoxon test FDR p-value 0.05 is depicted in FIG. 2. The peptides clusters indicate signals that rise and fall together (across samples). Note that signal groups that are relatively uncorrelated can be combined to provide approximately independent information in a screening or diagnostic panel.

Table 5 summarizes the results for polypeptides identified and associated with ovarian cancer.

TABLE 5

| | | | | Relative abundance | | | | SEQ |
|---|---|---|---|---|---|---|---|---|
| | | | Ions | | | Patient/ | | ID |
| Protein ID | Mass | Time | score | Patient | Control | Control | Peptide sequence | NO |
| gi\|4502027: albumin | 1638.928 | 35.15 | 107 | 4464112 | 2167818 | 2.059 | KVPQVSTPTLVEVSR | 25 |
| preproprotein | | | | | | | | |
| gi\|4502027: albumin | 998.51 | 66.58 | 43 | 104572 | 34562 | 3.026 | FYAPELLF | 26 |
| preproprotein | | | | | | | | |
| chymotryptic | | | | | | | | |
| gi\|4502027: albumin | 2044.088 | 52.9 | 95 | 5258510 | 2941741 | 1.788 | VFDEFKPLVEEPQNLIK | 27 |
| preproprotein | | | | | | | | |
| gi\|4502027: albumin | 1341.627 | 52.17 | 101 | 2030263 | 887045 | 2.289 | AVMDDFAAFVEK | 28 |
| preproprotein | | | | | | | | |
| gi\|4502027: albumin | 1395.797 | 42.7 | 88 | 67373 | 386 | 174.389 | KVPQVSTPTLVEV | 29 |
| preproprotein semi-tryptic | | | | | | | | |
| gi\|4502027: albumin | 1148.606 | 33.5 | 74 | 1537722 | 890088 | 1.728 | LVNEVTEFAK | 30 |
| preproprotein | | | | | | | | |
| gi\|4502027: albumin | 1404.715 | 49.93 | 48 | 395828 | 13855 | 28.57 | RHPYFYAPELL | 31 |
| preproprotein semi-tryptic | | | | | | | | |
| gi\|4502027: albumin | 1756.898 | 40 | 94 | 214352 | 270 | 792.761 | EDHVKLVNEVTEFAK | 32 |
| preproprotein semi-tryptic | | | | | | | | |
| gi\|4502027: albumin | 2098.007 | 52.79 | 63 | 102806 | 23125 | 4.446 | VFDEFKPLVEEPQNLIK | 33 |
| preproprotein | | | | | | | | |
| albumin ylyeiar | 952.498 | 44.1 | 47 | 192862 | 38163 | 5.054 | YLYEIAR | 34 |
| gi\|4502027: albumin | 1168.575 | 40.96 | 68 | 40211 | 0 | 40211 | NYAEAKDVFL | 35 |
| preproprotein semi-tryptic | | | | | | | | |
| gi\|4502027: albumin | 1302.737 | 38.63 | 107 | 54048 | 0 | 54048 | AEVSKLLVTDLTK | 36 |
| preproprotein semi-tryptic | | | | | | | | |
| gi\|4502027: albumin | 2070.104 | 61.11 | 76 | 607458 | 493748 | 1.23 | VFDEFKPLVEEPQNLIK | 37 |
| preproprotein + | | | | | | | | |
| modification | | | | | | | | |
| gi\|4826898: profilin 1 | 1212.623 | 32.14 | 65 | 32432 | 10349 | 3.134 | DSPSVWAAVPGK | 38 |
| gi\|34013530: acetyl | 1693.784 | 35.98 | 21 | 151617 | 0 | 151617 | SDKPDMAEIEKFDK | 39 |
| thymosin beta-4-like | | | | | | | | |
| protein 3 | | | | | | | | |
| gi\|4557321: | 1251.611 | 33.96 | | 68198 | 15597 | 4.373 | VQPYLDDFQK | 40 |
| apolipoprotein A-I | | | | | | | | |
| preproprotein | | | | | | | | |
| actin, ovary protein or | 1197.694 | 37.03 | 68 | 144812 | 40173 | 3.605 | AVFPSIVGRPR | 41 |
| periplakin | | | | | | | | |
| gi\|50363217: serine | 1014.605 | 41.43 | | 184715 | 44382 | 4.162 | SVLGQLGITK | 42 |
| proteinase inhibitor: | | | | | | | | |
| clade A: member 1 | | | | | | | | |
| gi\|4504345: alpha globin | 1294.674 | 39.31 | 103 | 15288 | 0 | 15288 | VADALTNAVAHVD | 43 |
| semi-tryptic | | | | | | | | |
| gi\|4506773: S100 A9 | 1494.743 | 29.14 | | 79406 | 0 | 79406 | LPHPDTLNQGEFK | 44 |
| mutant Gly−>Pro #27 | | | | | | | | |
| gi\|21614544: S100 | 1101.678 | 57.59 | 56 | 25074 | 0 | 25074 | QEFLILVIK | 45 |
| calcium-binding protein | | | | | | | | |
| A8 semi-tryptic | | | | | | | | |
| gi\|4506145: protease: | 2226.091 | 39.98 | | 14089 | 84546 | 0.167 | LGEHNIEVLEGNEQFIN | 46 |
| serine: 1 preproprotein | | | | | | | AAK | |
| gi\|4504349 beta globin | 2566.323 | 56.98 | | 110718 | 0 | 110718 | AHGKKVLGAFSDGLAHL | 47 |
| semi-tryptic | | | | | | | DNLKGTFA | |
| gi\|4504345: alpha globin | 1430.726 | 36.37 | 55 | 64382 | 11 | 5710.997 | AAHLPAEFTPAVHA | 48 |
| non-tryptic | | | | | | | | |
| | 1825.885 | 50.12 | | 307983 | 745 | 413.385 | | |
| gi\|21536452 mesotrypsin | 1534.741 | 41.66 | 91 | 8301 | 34169 | 0.243 | SLPYQVSLNSGSHF | 49 |
| isoform 2 preproprotein | | | | | | | | |
| non-tryptic | | | | | | | | |

### Example 4

### Analysis of Polypeptides Associated with Endometrial Cancer

Four groups of peptides previously associated with the presence of endometrial cancer were further analyzed. The groups included: albumin peptides; confidently-identified peptides; confidently-identified peptides+albumin peptides; and modified peptides.

### Data Source and Processing

The peptides to be further evaluated were identified by mass-to-charge (“mass”) and retention time (“time”). A total of 32 peptide signals selected from the data set for 306 mass spec runs (102 patient samples). Three LC-MS runs were performed for each patient sample. Peptide peak areas were normalized using the 80th percentile matching described in previous analyses. Peptides with zero peak areas were assumed to be below the limit of quantification (BQL). Zero areas were replaced with ½ the minimum reported peak area for the corresponding peptide. Peak areas were subsequently log 10 transformed, and averaged across the three runs for each patient. Thus, each patient contributes to the data analysis a single (log 10) average peak area for each peptide. Modified-to-unmodified peptide ratios were computed separately for each LC-MS run, after replacement of BQL values. Ratios were subsequently log 10 transformed and averaged for each patient.

### Statistical Modeling Approach

The primary method used for statistical model selection was the “lasso” with penalty factor chosen by leave-one-out cross-validation LOOCV (with minimum deviance criterion) (Tibshirani, R. (1996). “Regression Shrinkage and Selection via the Lasso” J. Roy. Statist. Soc. Ser. B., 58 (1): 267-288). The lasso selects a parsimonius set of predictors from a large set of potential predictors. It also provides a coefficient “shrinkage” estimation method that helps to prevent overfitting the training data, and improves prediction in independent test data sets. The lasso procedure is a penalized likelihood method in which the final number of selected predictors and their model coefficient shrinkage is controlled by a single penalty parameter. For all analyses described herein, the penalty parameter was selected using leave-one-out cross validation (LOOCV). LOOCV selects the statistical model which best predicts the outcome of each “hold-out” observation. A patient sample was selected, and temporarily held-out of the training data. A statistical model was fit to the training data, and the resulting parameter estimates were used to predict the value of the hold-out observation. The process was repeated for each sample (patient) in the data set. The penalty parameter with the best hold-out predictive performance was retained for fitting the entire data set. The criterion for evaluating hold-out predictive performance was logistic model deviance.

A secondary method of model selection, best subsets regression, was also used for the different peptide groups. This procedure exhibits very different operating characteristics than lasso, and is included to provide alternative modeling results. Best subsets examines all possible subsets of potential predictors, and selects the predictor set maximizing cross validation performance. This procedure tends to produce smaller statistical models (i.e., fewer predictors), but with larger estimated coefficients (no coefficient shrinkage). Thus, the fitted coefficients may over-predict in independent test data.

### Albumin Peptides

Six peptides from albumin were selected. The mass/time values for these are shown in Table 6. Peptides at 2097 and 2098 are modified versions of the 2044 peptide. These are believed to differ only in that they contain differing iron (Fe) isotopes. Similarly, the peptide with mass 1066 is likely a modified version of the 1012 peptide.

TABLE 6

| Mass | Time |
|---|---|
| 2098.01 | 52.99 |
| 2044.09 | 53.00 |
| 2097.01 | 52.97 |
| 1066.51 | 41.81 |
| 1012.59 | 42.11 |
| 1638.93 | 35.12 |

Among albumin peptides, an iron (Fe) modified peptide with mass 2098 was the single best predictor of endometrial cancer. Peptides at masses 1012, 1639, and 2044 were also useful in distinguishing endometrial cancer from control patient samples. The area under the receiver operating characteristic curve (AUC) for this four-predictor model was 0.90.

### Confidently-Identified Peptides

Confidently-identified peptides included 26 peptides, excluding the albumin peptides. A subset of 10 peptides were selected as predictors of endometrial cancer, these peptides included peptides with masses 1015 (SEQ ID NO:18), 1071 (SEQ ID NO:17). 1115 (SEQ ID NO:20), 1129 (SEQ ID NO:16), 1368, 1535 (SEQ ID NO:06), 1612 (SEQ ID NO:09), 2226 (SEQ ID NO:22), 3371, and 3662. This 10 predictor model exhibited an AUC of 0.96. Increases in peptides with masses 1015 (SEQ ID NO:18), 1071 (SEQ ID NO:17), 1129 (SEQ ID NO:16), 1368, 1612 (SEQ ID NO:09), and 3662 were associated with increased probability of endometrial cancer. Conversely, increased levels of peptides with masses 1535 (SEQ ID NO:06), 3371, 1115 (SEQ ID NO:20), and 2226 (SEQ ID NO:22), were associated with decreased probability. This collection of peptides exhibited a strong ability to discriminate control from endometrial cancer patient samples (p ˜10⁻¹¹), and an AUC of 0.96.

### Confidently-Identified Peptides+Albumin Peptides

Confidently-identified peptides+albumin peptides included the 26 confidently-identified peptides and the iron (Fe) modified peptide with mass 2098. This group was evaluated to distinguish endometrial cancer from control 10 peptides were selected with the following masses: 1015 (SEQ ID NO:18), 1071 (SEQ ID NO:17), 1115 (SEQ ID NO:20), 1230 (SEQ ID NO:14), 1535 (SEQ ID NO:06), 1612 (SEQ ID NO:09), 2098 (SEQ ID NO:01), 2996(0.461) (SEQ ID NO:24), 3371, and 3662. This model exhibited an observed AUC of 0.97. A second peptide selection strategy identified two of these predictors as most informative in predicting endometrial cancer. These were the peptides at masses 1071 (SEQ ID NO:17) and 3662. This two predictor model exhibited an AUC of 0.93. Increases in peptides with masses 1612 (SEQ ID NO:09), 3662, 1071 (SEQ ID NO:17), 1015 (SEQ ID NO:18), 2996 (mass 2996.461, time 53.07) (SEQ ID NO:24), and 2098 (SEQ ID NO:01), were associated with increased probability of endometrial cancer. Conversely, increased levels of 1535 (SEQ ID NO:06), 3371, 1230 (SEQ ID NO:14), and 1115 (SEQ ID NO:20) were associated with decreased probability. This collection of peptides exhibited a strong ability to discriminate control from endometrial cancer patient samples (p ˜10⁻¹²), and an AUC of 0.97.

### Modified Peptides

Modified peptides include a set of nine modified peptides were evaluated to identify potential predictors of endometrial cancer. Six of these nine peptides were selected in a logistic regression model. These included peptides at masses 1213 (SEQ ID NO:23), 1067 (SEQ ID NO:05), 2098 (SEQ ID NO:01), 2996 (2996.461) (SEQ ID NO:24), 3049 (SEQ ID NO:19), and 3662. The AUC for this six-predictor model was 0.95. An alternative subset selection strategy identified two of these predictors (3049, 3662) as being most informative in predicting endometrial cancer (AUC=0.93). Five modified peptides were selected for inclusion in the lasso logistic regression model. All were positively associated with increased probability of endometrial cancer. This collection of peptides is strongly associated with separation of control and endometrial cancer patient samples (p ˜10⁻¹²), and an AUC of 0.95.

### Example 5

### Analysis of Polypeptides Associated with Ovarian Cancer

Four groups of peptides previously associated with the presence of ovarian cancer were further analyzed. The groups included: albumin peptides; confidently-identified peptides; ANN peptides; and modified peptides. The AUC values ranged from 0.84 to 0.89. Many of the peptide signals were not observed in control samples, and observed in only a portion of ovarian cancer cases. It is unclear whether these peptides were absent from affected samples, or present but below detection limits

### Data Source and Processing

The peptides to be further evaluated were identified by mass-to-charge (“mass”) and retention time (“time”). A total of 36 peptide signals selected from the data set for 306 mass spec runs (102 patient samples). Three LC-MS runs were performed for each patient sample. Peptide peak areas were normalized using the 80th percentile matching described in previous analyses. Peptides with zero peak areas were assumed to be below the limit of quantification (BQL). Zero areas were replaced with ½ the minimum reported peak area for the corresponding peptide. Peak areas were subsequently log 10 transformed, and averaged across the three runs for each patient. Thus, each patient contributes to the data analysis a single (log 10) average peak area for each peptide. Modified-to-unmodified peptide ratios were computed separately for each LC-MS run, after replacement of BQL values. Ratios were subsequently log 10 transformed and averaged for each patient.

### Statistical Modeling Approach

The primary method used for statistical model selection was the “lasso” as described in Example 4.

### Albumin Peptides

Thirteen peptides from albumin were evaluated. These are listed in Table 7.

TABLE 7

| Mass | Time |
|---|---|
| 952.50 | 44.10 |
| 998.51 | 66.58 |
| 1148.61 | 33.50 |
| 1168.58 | 40.96 |
| 1302.74 | 38.63 |
| 1341.63 | 52.17 |
| 1395.80 | 42.70 |
| 1404.71 | 49.93 |
| 1638.93 | 35.15 |
| 1756.90 | 40.00 |
| 2044.09 | 52.90 |
| 2070.10 | 61.11 |
| 2098.01 | 52.79 |

The 13 peptides were evaluated as potential predictors. Although several of these peptides were related through post-translational modifications (e.g., peptides at 2097 and 2098 are modified versions of the 2044 peptide (SEQ ID NO:27)), combining peptides did not result in substantial improvement in predictive performance.

### Exploratory Analysis

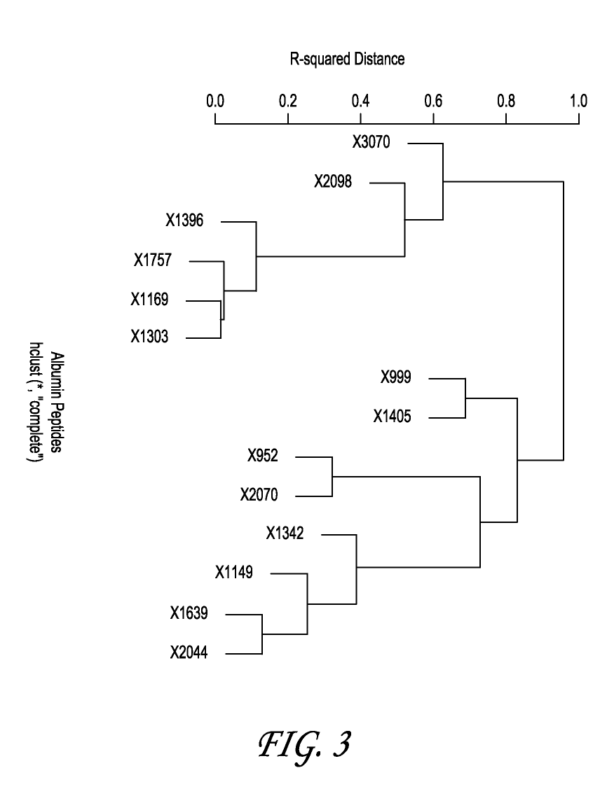

The relationships between albumin peptides' peak areas (loge o scale, patient means) were evaluated. FIG. 3 shows the results of peptide clustering, where clustering similarity is based on the squared correlation coefficient. Here, r² was between 0 and 1, with 1 denoting perfect linearity between two peptides. The “Rsquared Distance” was computed as 1-r² Thus, an R-squared distance near zero indicated nearly identical information in the two peptides. The cluster dendrogram shows that peptides at masses 1169 (SEQ ID NO:35), 1303 (SEQ ID NO:36), and 1757 (SEQ ID NO:32) were nearly co-linear. This meant that any one of these peptides contained almost the same information as the other two. As a consequence, only one of these three was useful for predictive modeling.

### Model Selection Results

The modeling approach selected six peptide signals for predicting ovarian cancer. These signals and their estimated coefficients are shown in Table 8.

TABLE 8

| Peptide | Coefficient | Odds Factor |
|---|---|---|
| X952 | 0.14 | 1.15 |
| X999 | 0.09 | 1.09 |
| X1405 | 0.44 | 1.56 |
| X2044 | 0.63 | 1.89 |
| X2098 | 0.15 | 1.17 |
| X3070 | 0.95 | 2.59 |

(Intercept −10.22)

The selected albumin peptides predicted ovarian cancer substantially better than random chance (p ˜10⁻⁶). The regression coefficients indicated that increases in any of the selected peptide peak areas were associated with increasing odds of ovarian cancer. The area under the receiver operating characteristic curve (AUC) was 0.85. This was better than random chance.

### Confidently Identified Peptides

### Modeling Results

Among the 6 confidently identified peptides with high association with disease, two were observed to be highly correlated (masses 1694 (SEQ ID NO:39) and 3070, r²=0.95). Thus, peptide 3070 was omitted from the model and the remaining 5 were included in the lasso penalized logistic regression model. These are listed in the Table 9, along with estimated coefficients and odds factors. The large coefficient and for peptide 1694 (SEQ ID NO:39) indicated numerical instability in the regression algorithm. This instability resulted from all control subjects having BDL values for peptide 1694 (SEQ ID NO:39) (same holds for peptide 3070).

TABLE 9

| Peptide | Coefficient | Odds Factor |
|---|---|---|
| X1015 | 1.07 | 2.93 |
| X1198 | −0.00 | 1.00 |
| X1213 | 0.52 | 1.69 |
| X1252 | 0.07 | 1.07 |
| X1694 | 26.95 | 504498674075.97 |

(Intercept −155.05)

Increases in peptides X1015 (SEQ ID NO:42), X1213 (SEQ ID NO:38), and X1694 (SEQ ID NO:39) were associated with increased probability of ovarian cancer. This collection of peptides exhibited a reasonable ability to discriminate control from ovarian cancer patient samples (p ˜10⁻⁵), and an AUC of 0.88.

### AAN Peptides

### Modeling Results

From eight AAN peptides, seven were selected for inclusion in the predictive model. Note that three peptides with very small, negative coefficients added little to the predictive ability of the model. These are listed in the Table 10, along with estimated coefficients and odds factors. The large positive coefficients resulted from having only BDL observations for the control patient samples; these peptides were detected only in samples from ovarian cancer patients, and not in controls.

TABLE 10

| Peptide | Coefficient | Odds Factor | Mass (Da) | Time of elution (mins) |
|---|---|---|---|---|
| X1283 | 3.02 | 20.49 | 1282.666 | 50.2 |
| X1311 | 13.87 | 1058779.20 | 1310.567 | 47.3 |
| X1420 | −0.41 | 0.66 | 1419.714 | 52.7 |
| X1452 | −0.25 | 0.78 | 1451.704 | 42.1 |
| X2155 | 11.95 | 154043.70 | 2155.062 | 60.5 |
| X2464 | −0.47 | 0.63 | 2462.297 | 57.6 |
| X2490 | 12.32 | 224569.29 | 2490.312 | 59.8 |

(Intercept −130.42)

Increases in peptides with masses X1311, X2155, and X2490, were associated with increased probability of ovarian cancer. This collection of peptides exhibited an ability to discriminate control from ovarian cancer patient samples (p ˜10⁻⁷), and an AUC of 0.89. The masses listed in Table 10 were measured to within 10 ppm of the true molecular weight. The polypeptides of Table 10 include non-standard peptide sequences, and some show similarity in their sequences.

To generate a reference curve, chromatography was performed on a 150 micron C18 column with various known human serum albumin tryptic peptides. The chromatographic retention time can be related to a series of well defined tryptic peptides from albumin as shown in Table 11. All chromatography was performed on a Thermo Electron Corporation Hypersil Gold C18 column with dimensions (mm) of 100×0.18 and a particle size of 3 microns (Thermo part number 25003-100265) using a flow rate of 1 microliter per minute and a gradient of acetonitrile in water with 0.2% formic acid. Table 11 provides the timescale representing an elution profile of known human serum albumin tryptic peptides.

TABLE 11

| SEQ | | Time of elution |
|---|---|---|
| ID NO: | Sequence | (mins) |
| 50 | VPQVSTPTLVEVSR | 37.4 |
| 51 | LVAASQAALGL | 42.1 |
| 52 | LVRPEVDVMCTAFHDNEETFLKK | 45.6 |
| 53 | QNCELFEQLGEYK | 48.6 |
| 54 | AAFTECCQAADKAACLLPK | 53.3 |
| 55 | AEFAEVSKLVTDLTK | 58.4 |
| 56 | SHCIAEVENDEMPADLPSLAADFVESK | 61.4 |
| 57 | NYAEAKDVFLGMFLYEYAR | 67.4 |

### Modified Peptides

Eight peptides with PTMs were selected for analysis. The mass and retention times for these peptides are shown in Table 12.

TABLE 12

| Mass | Time |
|---|---|
| 1294.67 | 39.31 |
| 1430.73 | 36.37 |
| 1494.74 | 29.14 |
| 1534.74 | 41.66 |
| 1825.88 | 50.12 |
| 2226.09 | 39.98 |
| 2566.32 | 56.98 |
| 3069.58 | 21.67 |

### Modeling Results

The lasso-CV procedure identified the following peptides in Table 13 for inclusion in the statistical model.

TABLE 13

| Peptide | Coefficient | Odds Factor |
|---|---|---|
| 1295 | 7.61 | 2016.67 |
| 1431 | 3.21 | 24.67 |
| 1535 | 0.47 | 0.62 |
| 1826 | 1.89 | 6.59 |
| 2226.1 | −0.77 | 0.46 |
| 3070 | 17.73 | 50216457.72 |

(Intercept −92.95)

Six modified peptides were selected for inclusion in the lasso logistic regression model. Four were positively associated with increased probability of ovarian cancer. This collection of peptides was strongly associated with separation of control and ovarian patient samples (p ˜10⁻⁶), and an AUC of 0.86.

### Example 6

### Analysis of Iron-Binding Peptides

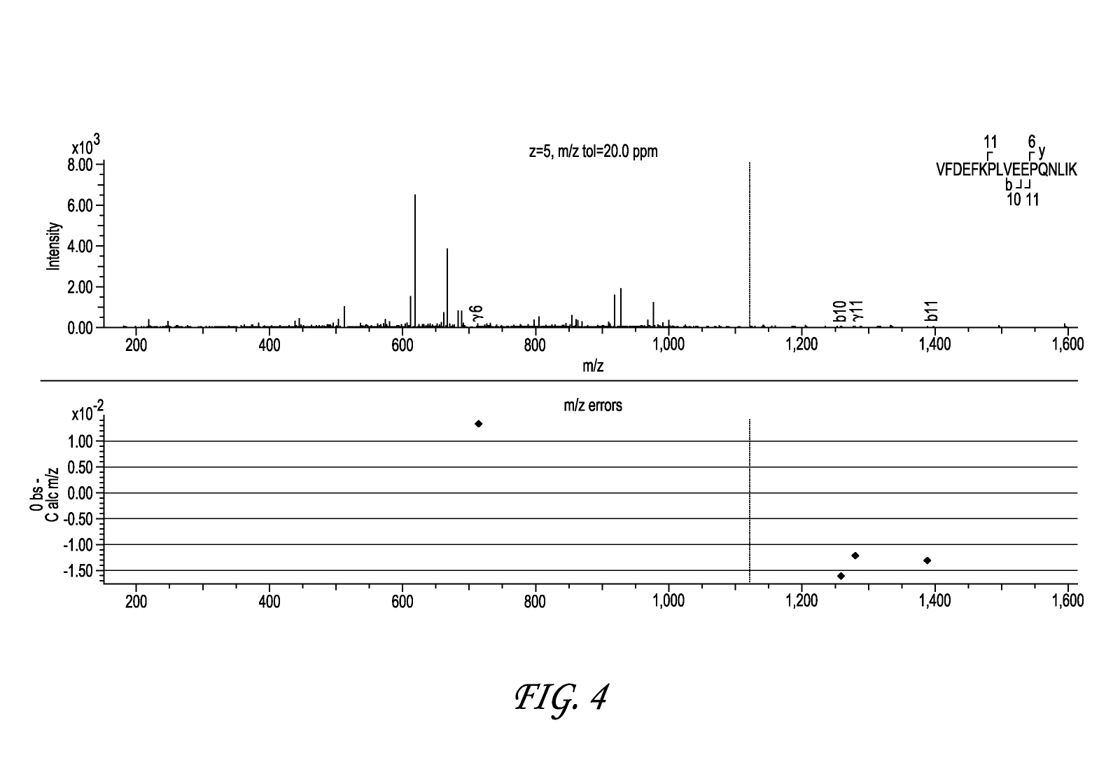

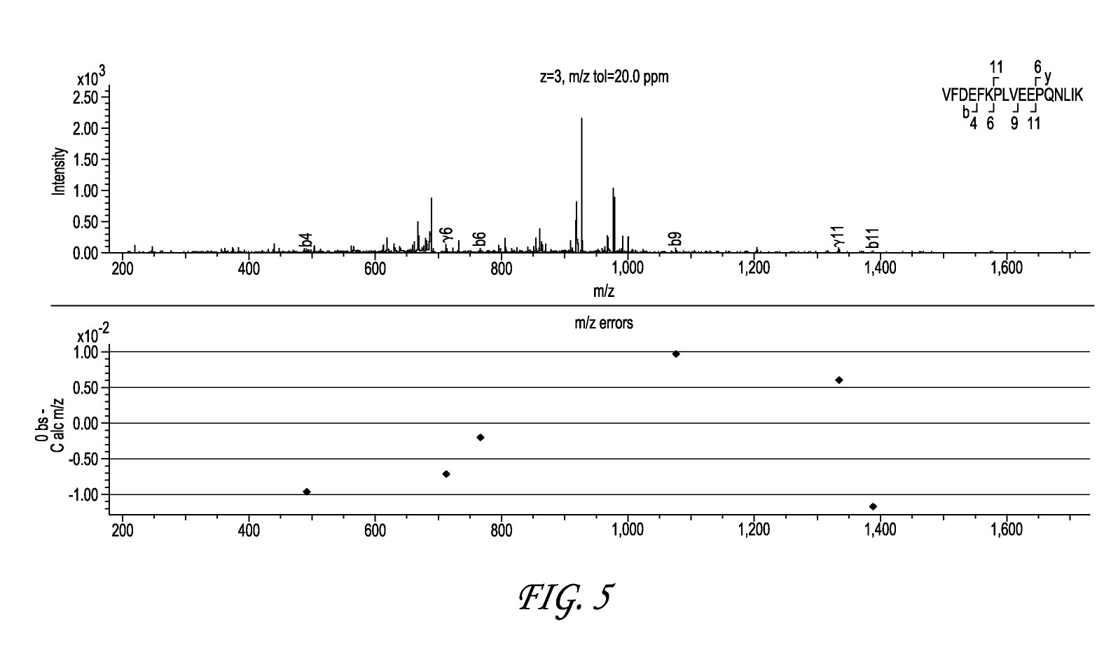

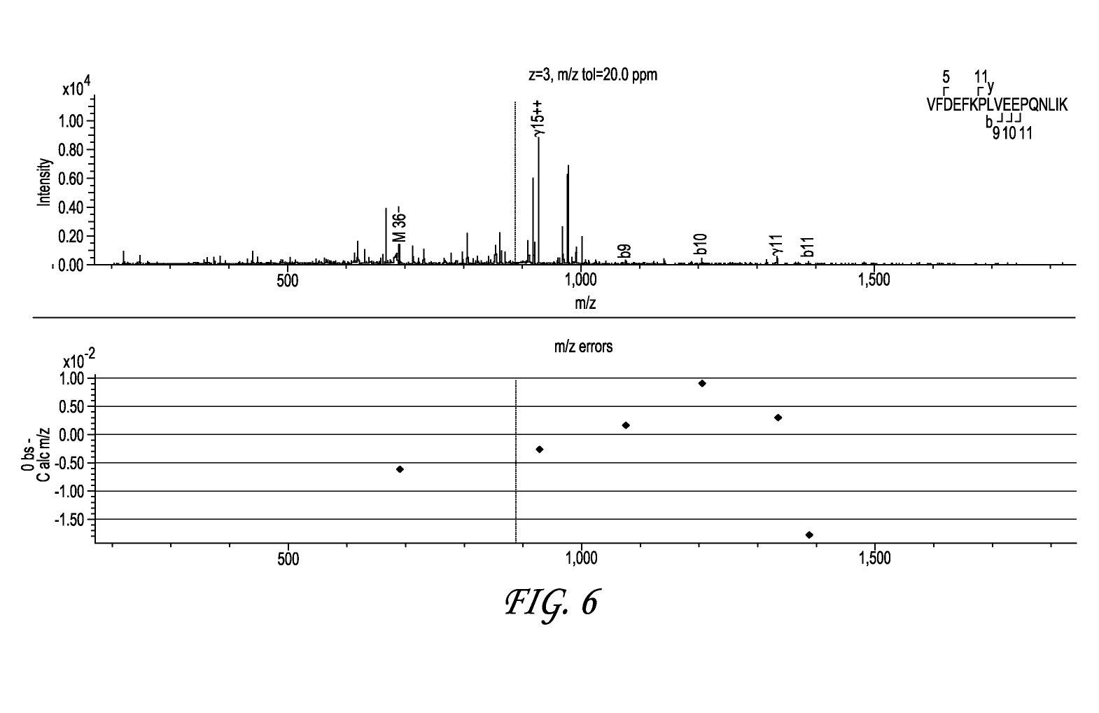

Iron-binding was further analyzed with regards to SEQ ID NO:01 using the BYONIC software package (Protein Metrics, San Carlos, Calif.). BYONIC software includes a search engine to identify modified polypeptides. In the BYONIC analysis, “Delta Score” is the difference in score from the top-scoring identification to an identification with a different base peptide; and “Delta Mod. Score” is the difference in score from the top-scoring identification to the next best identification that is different in any way, including modification localization. Sequences analyzed included SEQ ID NO:01 with an additional lysine residue at the N-terminus, and an additional glutamine residue at C-terminus Iron-modification are cited relative to SEQ ID NO:01. FIGS. 4, 5, and 6 show graphs of m/z vs. intensity (top panel) or observed calculated m/z, with regards to SEQ ID NO:01 iron-modified at residues E4, E10, and E11, respectively. Table 14 summarizes the results of the BYONIC analysis.

TABLE 14

| Iron- | | | | | | | |
|---|---|---|---|---|---|---|---|
| modi- | Obs/ | | Obs. | Pprm | | Delta | Delta |
| fication | m/z | z | MH | err. | Score | Score | Mod. Score |
| E11 | 700.343 | 3 | 2099.015 | 0.29 | 59.3 | 59.3 | 39.1 |
| E10 | 700.343 | 3 | 2099.015 | 0.03 | 39.8 | 39.9 | 0.0 |
| E11 | 525.509 | 4 | 2099.015 | 0.08 | 26.5 | 26.5 | 12.7 |
| E10 | 525.510 | 4 | 2099.017 | 1.24 | 15.0 | 15.0 | 10.6 |
| E4 | 420.609 | 5 | 2099.015 | 0.36 | 8.6 | 8.6 | 1.4 |
| None | 1023.053 | 2 | 2045.100 | 2.12 | 217.2 | 141.7 | 141.7 |

From the BYONIC analysis, SEQ ID NO:01 was found to be associated with iron at residues E4, E10, and E11. As indicated by higher Scores, iron was mostly likely to be associated with E10 and/or E11; iron association with E4 was much less favored.

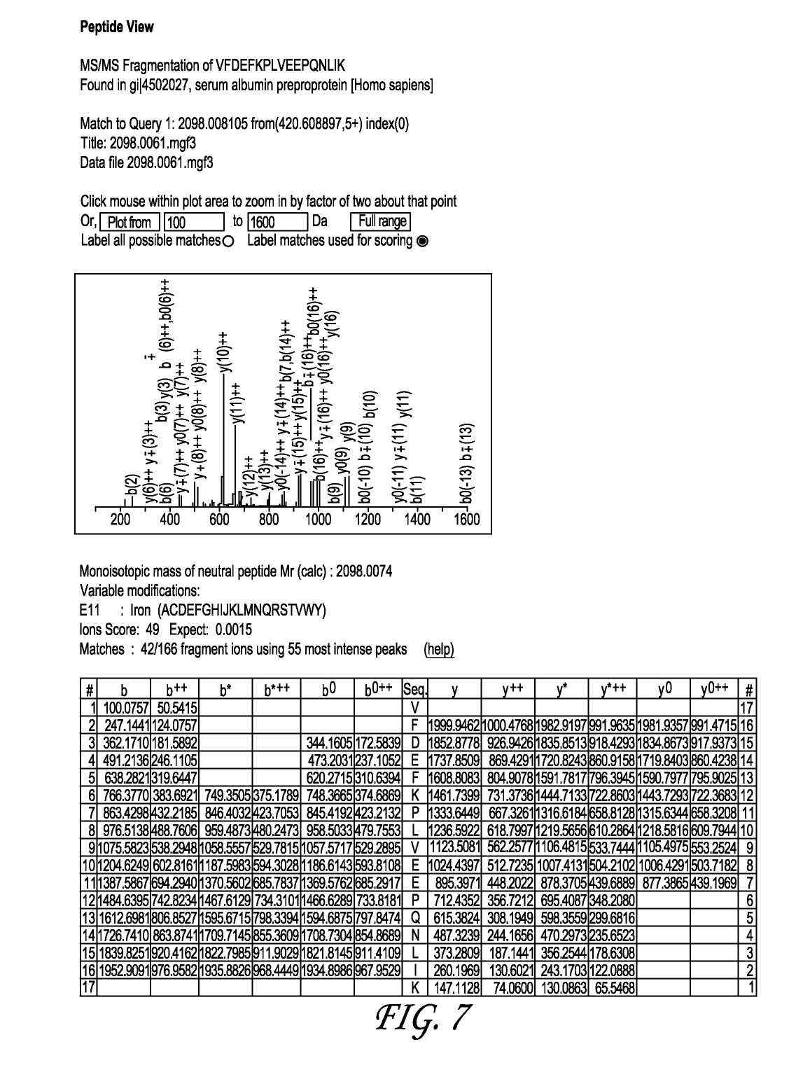

In additional to BYONIC analysis, iron association with SEQ ID NO:01 was further analyzed using MASCOT software. MASCOT identified an iron-association at E11 residue of SEQ ID NO:01. FIG. 7 provides an example MASCOT search result.

The term “comprising” as used herein is synonymous with “including,” “containing,” or “characterized by,” and is inclusive or open-ended and does not exclude additional, unrecited elements or method steps. As used herein “consisting essentially of” refers to a peptide or polypeptide which includes an amino acid sequence of the polypeptides provided herein, for example, SEQ ID NO.s 01-49, along with additional amino acids at the carboxyl and/or amino terminal ends where the additional amino acids do not materially alter the ability of the peptide or polypeptide to be diagnostically useful for the relevant type or types of cancer. For example, in some embodiments, a peptide or polypeptide “consisting essentially of” a particular sequence may include an amino acid sequence of the polypeptides provided herein, for example SEQ ID NO.s:01-49, along with no more than 1, no more than 2, no more than 3, no more than 4, no more than 5, no more than 6, no more than 7, no more than 8, no more than 9, or no more than 10 additional amino acid(s) at the carboxyl and/or amino terminal ends of a polypeptide provided herein, for example, one of SEQ ID NO.s: 01-49.

All numbers expressing quantities of ingredients, reaction conditions, and so forth used in the specification are to be understood as being modified in all instances by the term “about.” Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

The above description discloses several methods and materials of the present invention. This invention is susceptible to modifications in the methods and materials, as well as alterations in the fabrication methods and equipment. Such modifications will become apparent to those skilled in the art from a consideration of this disclosure or practice of the invention disclosed herein. Consequently, it is not intended that this invention be limited to the specific embodiments disclosed herein, but that it cover all modifications and alternatives coming within the true scope and spirit of the invention.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

What is claimed is:

1. A method for determining the presence, absence, progression, or stage of a cancer in a female subject comprising:
determining the level of at least one polypeptide or fragment thereof or the level of at least one nucleic acid encoding said at least one polypeptide or a fragment thereof in a sample from said subject, wherein the polypeptide is selected from the group consisting of a polypeptide comprising, consisting essentially of, or consisting of SEQ ID NO.s:06-24, and 38-49.

2. The method of claim 1, wherein the sample is obtained from the cervix, the vagina, or the posterior vaginal fornix.

3. The method of any one of claims 1-2, further comprising determining the level of at least one polypeptide comprising an amino acid sequence selected from the group consisting of a polypeptide comprising, consisting essentially of, or consisting of SEQ ID NO.s:01-05 and 25-37 or the level of at least one nucleic acids encoding said polypeptides or a fragment thereof.

4. The method of any one of claims 1-3, further comprising determining the level of at least one polypeptide comprising an amino acid sequence selected from the group consisting of a polypeptide comprising, consisting essentially of, or consisting of SEQ ID NO.s:01-49 or the level of at least one nucleic acids encoding said polypeptides or a fragment thereof.

5. The method of any one of claims 1-4, further comprising determining the level of at least two polypeptides comprising an amino acid sequence selected from the group consisting of a polypeptide comprising, consisting essentially of, or consisting of SEQ ID NO.s:01-49 or the level of at least two nucleic acids encoding said polypeptides or a fragment thereof.

6. The method of any one of claims 1-5, further comprising determining the level of at least three polypeptides comprising an amino acid sequence selected from the group consisting of a polypeptide comprising, consisting essentially of, or consisting of SEQ ID NO.s:01-49 or the level of at least three nucleic acids encoding said polypeptides or a fragment thereof.

7. The method of any one of claims 1-6, further comprising determining the level of at least five polypeptides comprising an amino acid sequence selected from the group consisting of a polypeptide comprising, consisting essentially of, or consisting of SEQ ID NO.s:01-49 or the level of at least five nucleic acids encoding said polypeptides or a fragment thereof.

8. The method of any one of claims 1-7, further comprising comparing the level of at least one polypeptide or the level of a nucleic acid encoding the polypeptide in a sample from the subject with the level of at least one polypeptide or the level of a nucleic acid encoding the polypeptide in a sample from a subject without the cancer.

9. The method of claim 8, wherein an increase in the level of said at least one polypeptide or fragment thereof or the level of said at least one nucleic acid encoding the polypeptide or a fragment thereof in a sample from said subject compared to the level of said at least one polypeptide or fragment thereof or the level of said at least one nucleic acid encoding said at least one polypeptide in a sample from said subject without cancer is indicative of the presence of the cancer in the subject.

10. The method of any one of claims 1-9, wherein at least one polypeptide is selected from the group consisting of a polypeptide comprising, consisting essentially of, or consisting of SEQ ID NO.s: 1-5, 7-19, 21, 23-45, and 47-48.

11. The method of any one of claims 1-10, wherein the cancer comprises endometrial cancer, wherein the polypeptide is selected from the group consisting of a polypeptide comprising, consisting essentially of, or consisting of SEQ ID NO.s: 1-5, 7-19, 21, 23-24.

12. The method of any one of claims 1-10, wherein the cancer comprises ovarian cancer, wherein the polypeptide is selected from the group consisting of a polypeptide comprising, consisting essentially of, or consisting of SEQ ID NO.s: 25-45, and 47-48.

13. The method of any one of claims 9-12, wherein at least a 3-fold increase in the level of the said at least one polypeptide or fragment thereof or the level of said at least one nucleic acid encoding the polypeptide or fragment thereof in a sample from said subject compared to the level of said at least one polypeptide or fragment thereof or the level of said at least one nucleic acid encoding the polypeptide or fragment thereof in a sample from said subject without cancer is indicative of the presence of the cancer in the subject.

14. The method of any one of claims 9-12, wherein at least a 5-fold increase in the level of the said at least one polypeptide or fragment thereof or the level of said at least one nucleic acid encoding the polypeptide or fragment thereof in a sample from said subject compared to the level of said at least one polypeptide or fragment thereof or the level of said at least one nucleic acid encoding the polypeptide or fragment thereof in a sample from said subject without cancer is indicative of the presence of the cancer in the subject.

15. The method of any one of claims 9-12, wherein at least a 10-fold increase in the level of the said at least one polypeptide or fragment thereof or the level of said at least one nucleic acid encoding the polypeptide or fragment thereof in a sample from said subject compared to the level of said at least one polypeptide or fragment thereof or the level of said at least one nucleic acid encoding the polypeptide or fragment thereof in a sample from said subject without cancer is indicative of the presence of the cancer in the subject.

16. The method of any one of claims 9-12, wherein at least a 100-fold increase in the level of the said at least one polypeptide or fragment thereof or the level of said at least one nucleic acid encoding the polypeptide or fragment thereof in a sample from said subject compared to the level of said at least one polypeptide or fragment thereof or the level of said at least one nucleic acid encoding the polypeptide or fragment thereof in a sample from said subject without cancer is indicative of the presence of the cancer in the subject.

17. The method of claim 8, wherein a decrease in the level of said at least one polypeptide or fragment thereof or the level of said at least one nucleic acid encoding the polypeptide or a fragment thereof in a sample from said subject compared to the level of said at least one polypeptide or fragment thereof or the level of said at least one nucleic acid encoding said at least one polypeptide in a sample from said subject without cancer is indicative of the presence of the cancer in the subject.

18. The method of any one of claims 1-8, and 17, wherein the at least one polypeptide is selected from the group consisting of a polypeptide comprising, consisting essentially of, or consisting of SEQ ID NO.s: 6, 20, 22, and 46.

19. The method of any one of claims 1-8, and 17-18, wherein the cancer comprises endometrial cancer, wherein the at least one polypeptide is selected from the group consisting of a polypeptide comprising, consisting essentially of, or consisting of SEQ ID NO.s: 6, 20, and 22.

20. The method of any one of claims 1-8, and 17-18, wherein the cancer comprises ovarian cancer, wherein the at least one polypeptide is SEQ ID NO.: 46.

21. The method of any one of claims 17-20, wherein at least a 3-fold decrease in the level of the said at least one polypeptide or fragment thereof or the level of said at least one nucleic acid encoding the polypeptide or fragment thereof in a sample from said subject compared to the level of said at least one polypeptide or fragment thereof or the level of said at least one nucleic acid encoding the polypeptide or fragment thereof in a sample from said subject without cancer is indicative of the presence of the cancer in the subject.

22. The method of any one of claims 17-20, wherein at least a 5-fold decrease in the level of the said at least one polypeptide or fragment thereof or the level of said at least one nucleic acid encoding the polypeptide or fragment thereof in a sample from said subject compared to the level of said at least one polypeptide or fragment thereof or the level of said at least one nucleic acid encoding the polypeptide or fragment thereof in a sample from said subject without cancer is indicative of the presence of the cancer in the subject.

23. The method of any one of claims 17-20, wherein at least a 10-fold decrease in the level of the said at least one polypeptide or fragment thereof or the level of said at least one nucleic acid encoding the polypeptide or fragment thereof in a sample from said subject compared to the level of said at least one polypeptide or fragment thereof or the level of said at least one nucleic acid encoding the polypeptide or fragment thereof in a sample from said subject without cancer is indicative of the presence of the cancer in the subject.

24. The method of any one of claims 1-23, wherein determining the level of said at least one polypeptide or fragment thereof comprises performing an immunoassay or a colorimetric assay.

25. The method of claim 24, wherein the immunoassay is selected from the group consisting of a Western blot, an enzyme linked immunoabsorbent assay (ELISA), and radioimmunoassay.

26. The method of any one of claims 1-23, wherein determining the level of said at least one polypeptide or fragment thereof comprises mass spectrometry.

27. The method of any one of claims 1-23, wherein determining the level of said at least one polypeptide or fragment thereof comprises:
applying said sample to a solid phase test strip or a flow-through strip comprising an agent which selectively binds to said at least one polypeptide or fragment thereof; and
detecting said polypeptide bound to said agent on said solid phase test strip or said flow-through strip.

28. The method of claim 1, wherein the cancer is a non-cervical cancer of the gynecological tract.

29. The method of claim 1, wherein the cancer is selected from the group consisting of endometrial cancer, and ovarian cancer.

30. The method of claim 1, wherein the cancer is selected from the group consisting of endometrial hyperplasia, endometrial hyperplasia with atypia, and non-invasive endometrial cancer.

31. The method of any one of claims 1-30, wherein the sample is obtained from a cervical pap specimen.

32. The method of any one of claims 1-31, wherein the sample is substantially free of cells.

33. The method of any one of claims 1-32, wherein said at least one polypeptide comprises a protein selected from the group consisting of mesotrypsin isoform 2, apolipoprotein A-I, transferring, alpha-1b-glycoprotein, hemopexin, alpha 2 globin, serine proteinase inhibitor: clade A: member 1, keratin 6C, profilin 1, periplakin, and calcium-binding protein A8 or fragment thereof.

34. The method of any one of claims 1-33, wherein the subject is human.

35. A kit for determining the presence, absence, progression, or stage of a cancer in a female subject comprising:
(a) a suitable diluent for irrigating the uterine cavity of the subject;
(b) a receptacle for collection of the diluted uterine fluid; and
(c) an agent that selectively binds to at least one polypeptide or nucleic acid encoding a polypeptide, wherein said polypeptide comprises an amino acid sequence selected from the group consisting of a polypeptide comprising, consisting essentially of, or consisting of SEQ ID NO.s:06-24, and 38-49.

36. The kit of claim 35, further comprising an agent that selectively binds to at least one polypeptide or nucleic acid encoding a polypeptide, wherein said polypeptide is selected from the group consisting of a polypeptide comprising, consisting essentially of, or consisting of SEQ ID NO.s:01-05 and 25-37.

37. The kit of any one of claims 35-36, further comprising an agent that selectively binds to at least one polypeptide or nucleic acid encoding a polypeptide, wherein said polypeptide is selected from the group consisting of a polypeptide comprising, consisting essentially of, or consisting of SEQ ID NO.s:01-49.

38. The kit of any one of claims 35-37, further comprising at least three agents that each selectively bind to a different polypeptide or a nucleic acid encoding said polypeptide.

39. The kit of any one of claims 35-38, further comprising at least five agents that each selectively bind to a different polypeptide or a nucleic acid encoding said polypeptide

40. The kit of any one of claims 35-39, wherein the agent comprises an antibody or antigen-binding fragment thereof.

41. The kit of any one of claims 35-40, wherein said at least one polypeptide comprises a protein selected from the group consisting of mesotrypsin isoform 2, apolipoprotein A-I, transferring, alpha-1b-glycoprotein, hemopexin, alpha 2 globin, serine proteinase inhibitor: clade A: member 1, keratin 6C, profilin 1, periplakin, and calcium-binding protein A8 or fragment thereof.

42. A kit comprising an agent which selectively binds to at least one polypeptide comprising an amino acid sequence selected from the group consisting of a polypeptide comprising, consisting essentially of, or consisting of SEQ ID NO.s:06-24, and 38-49 or a fragment thereof, wherein said agent is attached to a solid support.

43. The kit of claim 42, further comprising an agent that selectively binds to at least one polypeptide or nucleic acid encoding a polypeptide, wherein said polypeptide is selected from the group consisting of a polypeptide comprising, consisting essentially of, or consisting of SEQ ID NO.s:01-05 and 25-37.

44. The kit of any one of claims 42-43, further comprising an agent that selectively binds to at least one polypeptide or nucleic acid encoding a polypeptide, wherein said polypeptide is selected from the group consisting of a polypeptide comprising, consisting essentially of, or consisting of SEQ ID NO.s:01-49.

45. The kit of any one of claims 42-44, wherein a plurality of agents that bind to different polypeptides comprising an amino acid sequence selected from the group consisting of a polypeptide comprising, consisting essentially of, or consisting of SEQ ID NO.s:01-49 or a fragment thereof are attached to said solid support.

46. The kit of claim 45, wherein the solid support comprises a solid phase test strip or a flow-through test strip.

47. The kit of any one of claims 42-46, further comprising a detectable agent which selectively binds to said polypeptide.

48. The kit of any one of claims 42-46, wherein said at least one polypeptide comprises a protein selected from the group consisting of mesotrypsin isoform 2, apolipoprotein A-I, transferring, alpha-1b-glycoprotein, hemopexin, alpha 2 globin, serine proteinase inhibitor: clade A: member 1, keratin 6C, profilin 1, periplakin, and calcium-binding protein A8 or fragment thereof.

49. A kit comprising an agent which selectively binds to at least one nucleic acid encoding a polypeptide comprising an amino acid sequence selected from the group consisting of a polypeptide comprising, consisting essentially of, or consisting of SEQ ID NO.s:06-24, and 38-49 or a fragment thereof, wherein said agent is attached to a solid support.

50. The kit of claim 49, further comprising an agent that selectively binds to at least one polypeptide or nucleic acid encoding a polypeptide, wherein said polypeptide is selected from the group consisting of a polypeptide comprising, consisting essentially of, or consisting of SEQ ID NO.s:01-05 and 25-37.

51. The kit of any one of claims 49-50, further comprising an agent that selectively binds to at least one polypeptide or nucleic acid encoding a polypeptide, wherein said polypeptide is selected from the group consisting of a polypeptide comprising, consisting essentially of, or consisting of SEQ ID NO.s:01-49.

52. The kit of any one of claims 49-51, wherein a plurality of agents that bind to nucleic acids encoding different polypeptides comprising an amino acid sequence selected from the group consisting of a polypeptide comprising, consisting essentially of, or consisting of SEQ ID NO.s:01-49 or a fragment thereof are attached to said solid support.

53. The kit of claim 52, wherein the solid support comprises a solid phase test strip or a flow-through test strip.

54. The kit of any one of claims 49-53, further comprising a detectable agent which selectively binds to said polypeptide.

55. The kit of any one of claims 49-54, wherein said at least one polypeptide comprises a protein selected from the group consisting of mesotrypsin isoform 2, apolipoprotein A-I, transferring, alpha-1b-glycoprotein, hemopexin, alpha 2 globin, serine proteinase inhibitor: clade A: member 1, keratin 6C, profilin 1, periplakin, and calcium-binding protein A8 or fragment thereof.

56. The kit of any one of claims 49-55, wherein the cancer is selected from the group consisting of endometrial cancer, and ovarian cancer.

57. The kit of any one of claims 49-56, wherein the cancer is selected from the group consisting of endometrial hyperplasia, endometrial hyperplasia with atypia, and non-invasive endometrial cancer.

58. An isolated polypeptide consisting essentially of an amino acid sequence selected from the group consisting of a polypeptide comprising, consisting essentially of, or consisting of SEQ ID NO.s:06-24, and 38-49 or a fragment thereof, wherein said polypeptide is differentially expressed in cancer.

59. An isolated nucleic acid encoding a polypeptide consisting essentially of an amino acid sequence selected from the group consisting of a polypeptide comprising, consisting essentially of, or consisting of SEQ ID NO.s:06-24, and 38-49 or a fragment thereof, wherein said polypeptide is differentially expressed in cancer.

60. An isolated polypeptide consisting of an amino acid sequence selected from the group consisting of a polypeptide comprising, consisting essentially of, or consisting of SEQ ID NO.s:06-24, and 38-49 or a fragment thereof, wherein said polypeptide is differentially expressed in cancer.

61. An isolated nucleic acid encoding a polypeptide consisting of an amino acid sequence selected from the group consisting of a polypeptide comprising, consisting essentially of, or consisting of SEQ ID NO.s:06-24, and 38-49 or a fragment thereof, wherein said polypeptide is differentially expressed in cancer.

62. An isolated agent that selectively binds to an isolated polypeptide consisting essentially of an amino acid sequence selected from the group consisting of a polypeptide comprising, consisting essentially of, or consisting of SEQ ID NO.s:06-24, and 38-49 or a fragment thereof, wherein said polypeptide is differentially expressed in cancer.

63. The isolated agent of claim 62, wherein the agent comprises an antibody or antigen-binding fragment thereof.

64. An isolated agent that selectively binds to an isolated polypeptide consisting of an amino acid sequence selected from the group consisting of a polypeptide comprising, consisting essentially of, or consisting of SEQ ID NO.s:06-24, and 38-49 or a fragment thereof, wherein said polypeptide is differentially expressed in cancer.

65. The isolated agent of claim 64, wherein the agent comprises an antibody or antigen-binding fragment thereof.


SEQUENCE LISTING (57 sequences)

SEQ ID NO: 1 - protein, 17 aa, Homo Sapiens
  PEPTIDE (1)...(15): albumin 397-413
VFDEFKPLVEEPQNLIK

SEQ ID NO: 2 - protein, 11 aa, Homo Sapiens
  PEPTIDE (1)...(10): albumin preproprotein
LVAASQAALGL

SEQ ID NO: 3 - protein, 17 aa, Homo Sapiens
  PEPTIDE (1)...(15): albumin preproprotein
VFDEFKPLVEEPQNLIK

SEQ ID NO: 4 - protein, 15 aa, Homo Sapiens
  PEPTIDE (1)...(15): albumin preproprotein
KVPQVSTPTLVEVSR

SEQ ID NO: 5 - protein, 11 aa, Homo Sapiens
  PEPTIDE (1)...(10): albumin 599-609
LVAASQAALGL

SEQ ID NO: 6 - protein, 14 aa, Homo Sapiens
  PEPTIDE (1)...(10): mesotrypsin isoform 2 preproprotein non-tryptic
SLPYQVSLNSGSHF

SEQ ID NO: 7 - protein, 9 aa, Homo Sapiens
  PEPTIDE (1)...(5): apolipoprotein A-I preproprotein
WQEEMELYR

SEQ ID NO: 8 - protein, 12 aa, Homo Sapiens
  PEPTIDE (1)...(10): apolipoprotein A-I preproprotein
VSFLSALEEYTK

SEQ ID NO: 9 - protein, 14 aa, Homo Sapiens
  PEPTIDE (1)...(10): apolipoprotein A-I preproprotein
LLDNWDSVTSTFSK

SEQ ID NO: 10 - protein, 8 aa, Homo Sapiens
  PEPTIDE (1)...(5): transferrin
YLGEEYVK

SEQ ID NO: 11 - protein, 22 aa, Homo Sapiens
  PEPTIDE (1)...(20): transferrin
SMGGKEDLIWELLNQAQEHFGK

SEQ ID NO: 12 - protein, 13 aa, Homo Sapiens
  PEPTIDE (1)...(10): apolipoprotein A-I preproprotein
DYVSQFEGSALGK

SEQ ID NO: 13 - protein, 11 aa, Homo Sapiens
  PEPTIDE (1)...(10): transferrin
SASDLTWDNLK

SEQ ID NO: 14 - protein, 11 aa, Homo Sapiens
  PEPTIDE (1)...(10): apolipoprotein A-I preproprotein
QGLLPVLESFK

SEQ ID NO: 15 - protein, 12 aa, Homo Sapiens
  PEPTIDE (1)...(10): alpha-1b-glycoprotein
RLETPDFQLFKN

SEQ ID NO: 16 - protein, 8 aa, Homo Sapiens
  PEPTIDE (1)...(5): hemopexin precursor
RLWWLDLK

SEQ ID NO: 17 - protein, 9 aa, Homo Sapiens
  PEPTIDE (1)...(5): alpha 2 globin
MFLSFPTTK

SEQ ID NO: 18 - protein, 10 aa, Homo Sapiens
  PEPTIDE (1)...(10): serine proteinase inhibitor clade A member 1
SVLGQLGITK

SEQ ID NO: 19 - protein, 29 aa, Homo Sapiens
  PEPTIDE (1)...(25): alpha-globin 2995.481Fmod
VADALTNAVAHVDDMPNALSALSDLHAHK

SEQ ID NO: 20 - protein, 10 aa, Homo Sapiens
  PEPTIDE (1)...(10): keratin 6C
LEGLEDALQK

SEQ ID NO: 21 - protein, 10 aa, Homo Sapiens
  PEPTIDE (1)...(10): serine proteinase inhibitor clade A member 1
LSITGTYDLK

SEQ ID NO: 22 - protein, 20 aa, Homo Sapiens
  PEPTIDE (1)...(20): protease serine 1 preproprotein
LGEHNIEVLEGNEQFINAAK

SEQ ID NO: 23 - protein, 11 aa, Homo Sapiens
  PEPTIDE (1)...(10): apolipoprotein A-I preproprotein
QGLLPVLESFK

SEQ ID NO: 24 - protein, 29 aa, Homo Sapiens
  PEPTIDE (1)...(25): alpha 2 globin
VADALTNAVAHVDDMPNALSALSDLHAHK

SEQ ID NO: 25 - protein, 15 aa, Homo Sapiens
  PEPTIDE (1)...(15): albumin preproprotein
KVPQVSTPTLVEVSR

SEQ ID NO: 26 - protein, 8 aa, Homo Sapiens
  PEPTIDE (1)...(5): albumin preproprotein chymotryptic
FYAPELLF

SEQ ID NO: 27 - protein, 17 aa, Homo Sapiens
  PEPTIDE (1)...(15): albumin preproprotein
VFDEFKPLVEEPQNLIK

SEQ ID NO: 28 - protein, 12 aa, Homo Sapiens
  PEPTIDE (1)...(10): albumin preproprotein
AVMDDFAAFVEK

SEQ ID NO: 29 - protein, 13 aa, Homo Sapiens
  PEPTIDE (1)...(10): albumin preproprotein semi-tryptic
KVPQVSTPTLVEV

SEQ ID NO: 30 - protein, 10 aa, Homo Sapiens
  PEPTIDE (1)...(10): albumin preproprotein
LVNEVTEFAK

SEQ ID NO: 31 - protein, 11 aa, Homo Sapiens
  PEPTIDE (1)...(10): albumin preproprotein semi-tryptic
RHPYFYAPELL

SEQ ID NO: 32 - protein, 15 aa, Homo Sapiens
  PEPTIDE (1)...(15): albumin preproprotein semi-tryptic
EDHVKLVNEVTEFAK

SEQ ID NO: 33 - protein, 17 aa, Homo Sapiens
  PEPTIDE (1)...(15): albumin preproprotein
VFDEFKPLVEEPQNLIK

SEQ ID NO: 34 - protein, 7 aa, Homo Sapiens
  PEPTIDE (1)...(5): albumin ylyeiar
YLYEIAR

SEQ ID NO: 35 - protein, 10 aa, Homo Sapiens
  PEPTIDE (1)...(10): albumin preproprotein semi-tryptic
NYAEAKDVFL

SEQ ID NO: 36 - protein, 13 aa, Homo Sapiens
  PEPTIDE (1)...(10): albumin preproprotein semi-tryptic
AEVSKLLVTDLTK

SEQ ID NO: 37 - protein, 17 aa, Homo Sapiens
  PEPTIDE (1)...(15): albumin preproprotein + modification
VFDEFKPLVEEPQNLIK

SEQ ID NO: 38 - protein, 12 aa, Homo Sapiens
  PEPTIDE (1)...(10): profilin 1
DSPSVWAAVPGK

SEQ ID NO: 39 - protein, 14 aa, Homo Sapiens
  PEPTIDE (1)...(10): acetyl thymosin beta-4-like protein 3
SDKPDMAEIEKFDK

SEQ ID NO: 40 - protein, 10 aa, Homo Sapiens
  PEPTIDE (1)...(10): apolipoprotein A-I preproprotein
VQPYLDDFQK

SEQ ID NO: 41 - protein, 11 aa, Homo Sapiens
  PEPTIDE (1)...(10): actin, ovary protein or periplakin
AVFPSIVGRPR

SEQ ID NO: 42 - protein, 10 aa, Homo Sapiens
  PEPTIDE (1)...(10): serine proteinase inhibitor clade A member 1
SVLGQLGITK

SEQ ID NO: 43 - protein, 13 aa, Homo Sapiens
  PEPTIDE (1)...(10): alpha globin semi-tryptic
VADALTNAVAHVD

SEQ ID NO: 44 - protein, 13 aa, Homo Sapiens
  PEPTIDE (1)...(10): S100 A9 mutant Gly->Pro #27
LPHPDTLNQGEFK

SEQ ID NO: 45 - protein, 9 aa, Homo Sapiens
  PEPTIDE (1)...(5): S100 calcium-binding protein A8 semi-tryptic
QEFLILVIK

SEQ ID NO: 46 - protein, 20 aa, Homo Sapiens
  PEPTIDE (1)...(20): protease serine 1 preproprotein
LGEHNIEVLEGNEQFINAAK

SEQ ID NO: 47 - protein, 25 aa, Homo Sapiens
  PEPTIDE (1)...(25): beta globin semi-tryptic
AHGKKVLGAFSDGLAHLDNLKGTFA

SEQ ID NO: 48 - protein, 14 aa, Homo Sapiens
  PEPTIDE (1)...(10): alpha globin non-tryptic
AAHLPAEFTPAVHA

SEQ ID NO: 49 - protein, 14 aa, Homo Sapiens
  PEPTIDE (1)...(10): mesotrypsin isoform 2 preproprotein non-tryptic
SLPYQVSLNSGSHF

SEQ ID NO: 50 - protein, 14 aa, Homo sapiens
VPQVSTPTLVEVSR

SEQ ID NO: 51 - protein, 11 aa, Homo sapiens
LVAASQAALGL

SEQ ID NO: 52 - protein, 23 aa, Homo sapiens
LVRPEVDVMCTAFHDNEETFLKK

SEQ ID NO: 53 - protein, 13 aa, Homo sapiens
QNCELFEQLGEYK

SEQ ID NO: 54 - protein, 19 aa, Homo sapiens
AAFTECCQAADKAACLLPK

SEQ ID NO: 55 - protein, 15 aa, Homo sapiens
AEFAEVSKLVTDLTK

SEQ ID NO: 56 - protein, 27 aa, Homo sapiens
SHCIAEVENDEMPADLPSLAADFVESK

SEQ ID NO: 57 - protein, 19 aa, Homo sapiens
NYAEAKDVFLGMFLYEYAR